(19) United States
(12) Patent Application Publication
(10) Pub. No.: US 2023/0235383 A1
(43) Pub. Date: Jul. 27, 2023

(54) DNA NANOARRAYS

(71) Applicant: Massachusetts Institute of Technology, Cambridge, MA (US)

(72) Inventors: Eyal Perry, Cambridge, MA (US); Jan Tiepelt, Cambridge, MA (US); Joseph Jacobson, Newton, MA (US)

(73) Assignee: Massachusetts Institute of Technology, Cambridge, MA (US)

(21) Appl. No.: 18/168,385

(22) Filed: Feb. 13, 2023

Related U.S. Application Data
(60) Provisional application No. 63/289,516, filed on Dec. 14, 2021.

(51) Int. Cl. C12Q 1/6837; C12Q 1/6806; C12Q 1/6874

(52) U.S. Cl. CPC C12Q 1/6837; C12Q 1/6806; C12Q 1/6874

(57) ABSTRACT

A DNA nanoarray includes a milliscale chip substrate; a microscale binder spot having a uniform surface bound to the substrate; and immobilized oligonucleotide sequences, each linked to the binder. The immobilized oligonucleotide sequences form a monolayer, each having a length that guarantees within a statistical certainty that the immobilized oligonucleotide sequences are each unique. A method of producing the DNA nanoarray includes providing a streptavidin-coated substrate; patterning the substrate by photolithography; and immobilizing biotin-tagged oligonucleotides on the patterned surface. The oligonucleotides each have a unique string of bases. The patterned surface has an array of microscale spots with active streptavidin binding sites. Immobilization includes applying a solution containing the oligonucleotides to the microscale spots; applying a buffer over the patterned surface; and washing the patterned surface in buffered saline solution. Bits and/or spatial patterns may be stored the DNA nanoarray, then read and/or visualized.

### CROSS-REFERENCE TO RELATED APPLICATION

This application claims the benefit of priority of U.S. provisional application No. 63/289,516, filed Dec. 14, 2021, the contents of which are herein incorporated by reference.

### BACKGROUND OF THE INVENTION

The present invention relates to DNA synthesis and sequencing and, more particularly, to DNA nanoarrays.

DNA Nanotechnology

Deoxyribonucleic Acid (DNA) is composed of chemical building blocks called nucleotides. Each nucleotide has a sugar group, a phosphate group, and one of four nitrogen nucleobases: Cytosine [C], Guanine [G], Adenine [A] or Thymine [T]. Nucleotides are linked into chains (strands), with the phosphate and sugar groups alternating. Two DNA strands bind together with hydrogen bonds according to specific base-pairing rules: A binds with T, and C binds with G. This unique feature enables protein molecular machines, known as enzymes, to replicate long chains of DNA with extremely high precision. The fault-tolerant replication process holds the key to all life forms, as we are currently aware of, where DNA is the information molecule.

In 1982, inspired by the symmetry and spatial arrangements of a school of fish in M. C. Escher's wood engraving Depth (see FIG. 1A), Nadrian Seeman was the first to hypothesize that DNA could be used as a programmable nanoscale building material [1], leading to the birth of DNA nanotechnology. The proof-of-concept came from the simple idea that base-pairing rules can be used for more than a duplex of strands. Meaning, if four strands were to be designed and synthesized to share a similarity—where each strand is complementary to half of another strand—a crossbar-like junction will be formed by self-assembly. A simple junction structure can then be extended as “tiles” to form a 2D lattice, or a 3D cube. See FIGS. 1B and 1C.

In 2006, a new DNA assembly approach—now called “DNA Origami”—was invented by Paul Rothemund with Erik Winfree, using a single-stranded 7.3-kb-long genome (extracted from a natural-occurring M13 bacteriophage) as a scaffold, linked together at ˜200 points using short synthetic DNA strands (“staples”), to fold planar, arbitrarily shaped, 2D objects with length scales around 100 nm [5]. Since then, DNA origami has been developed to form complex nanoscale 2D and 3D structures (see FIG. 2). Currently, DNA origami and DNA tile assembly are commonly used for self-assembled construction of nanostructures. The nanoscale devices fabricated by these techniques have been utilized for a wide range of applications, including nanoplasmonics, nanophotonics, biosensing, and drug delivery. For an in-depth review on design strategies and various applications of DNA nanotechnology, see recent reviews by Seeman and Sleiman [6] and Wang et al. [4].

DNA Synthesis and Sequencing

The core of all DNA-based technologies is our ability to engineer and manipulate nucleic acids. The process of “reading” DNA fragments into a digital file (encoded as a string of A/C/G/T's) is known as sequencing. The method of “writing” physical DNA given a digital input is known as synthesis. DNA sequencing has been revolutionized over the past two decades, mostly thanks to the invention of next-generation sequencing (NGS) technology, leading to a drop in the price per base by six orders of magnitude [7]. Nature, by evolution, had granted us extremely efficient and high-fidelity enzymes to synthesize DNA given a template strand. However, we are currently unaware of any efficient natural system that can reliably synthesize long DNA strands in a template-independent (de novo) matter. Therefore, the cost of de novo synthesis has only slightly declined over the last few decades and continues to be significantly higher than the cost to sequence. (FIG. 3)

The gold standard method for de novo DNA synthesis is phosphoramidite chemistry, a powerful technique that has matured over several decades [8]. For each base in the desired sequence, a cycle of four chemical steps (detritylation, coupling, capping, and oxidation) is executed to extend an existing DNA strand on solid support. The chemical synthesis process is sequential per base yet is simultaneous for quadrillions or more DNA strands on the surface. The method produces accurate DNA sequences up to 200 bases, after which the yield drops exponentially. Interestingly, despite the high cost of synthesizing long DNA sequences, the phosphoramidite method allows synthesis of an enormous library of unique short strands at a fixed cost, using mixed bases (also known as wobbles or randomized bases). In each coupling step, instead of adding a single nucleoside (a nucleotide without the phosphate group), a mixture of nucleosides with different bases is added. By a random chemical process, the specific location on the synthesized strand could now contain any of the mixed bases. Since this process occurs for a vast amount of strands concurrently, a sequence library is generated. The synthesis cost per mixed base is equal to the cost per single base. According to degenerate bases nomenclature [9], the letter ‘N’ represents “any base”. Synthesizing ten consecutive Ns would result in a library that has 4¹⁰≈10⁶ unique, random sequences. In 2020, Meiser et al. demonstrated how this strategy can be employed as a true random number generator [10].

DNA Microarrays

DNA microarrays (also known as DNA chips) are an invaluable tool in many modern genomic studies. Essentially, microarrays are a group of DNA technologies where nucleic acid sequences are either deposited or synthesized in a 2-D (or sometimes 3-D) array, such that the sequences are immobilized to a surface (chip). Typically, each spot on the array (also known as a feature) is composed of >picomoles (>10¹² copies) of oligonucleotides sharing the same sequence. Each spot acts as a probe and the microarray serves as a sensing platform. See FIG. 4. By suspending a mixture of unknown DNA content on the chip, fragments of the target DNA will hybridize to the probes, according to base-pairing rules. Each hybridization event can be measured (usually by fluorescence) such that the DNA content and sequence are estimated. With the recent availability of reference genomes (Homo sapiens included), DNA microarrays are commonly used as fast, affordable genomic biosensors, in a process called genomic resequencing. DNA microarrays design, fabrication, and applications have been extensively reviewed in [12, 13].

A single probe can detect whether a single sequence (typically short, 25-100 bases long) exists in a target solution. By massively multiplexing to a large number of probes (some overlapping by sequence), the target solution can be tested for arbitrarily larger sequences. Therefore, there is a constant demand to maximize the number of unique probes on the surface, typically by minimizing the feature size. Fabrication techniques, that were originally developed for microelectronics, are now used in combination with chemical synthesis to create microscale features [15]. Currently, the smallest feature size for DNA microarrays is 5 μm (Affymetrix GeneChip® Human Gene 2.0ST). While micro- and nano-fabrication methods exist to generate <1 μm features, nanoscale probes face other significant challenges for detection such as a binding limit due to the law of mass action [16], and importantly, the diffraction limit, which restricts the ability of any optical instrument to distinguish between two features separated by a lateral distance less than approximately half the wavelength of light used for imaging.

DNA Nanoarrays

Over the last few decades, the field of nanofabrication has been revolutionized by technologies such as electron beam lithography and nanoimprint lithography, as previously reviewed in [17]. Nonetheless, it is still exceptionally expensive to dynamically pattern at the nanometer regime, where the lowest cost of a deep sub-micron lithography tool is on the order of >$100K. Furthermore, nanoscale placement of multiple uniquely addressable features, such as a variety of proteins, DNA probes or small-molecule ligands using current top-down approaches remains a challenging task.

Artificial DNA nanostructures, such as DNA origami, have great potential as nanoarray platforms. DNA can be folded from a long single-stranded viral DNA to complex nanostructures with the help of more than 200 helper strands called “staples”. Specifically, DNA can be folded to a 100 nm wide flat surface. Each of the ˜200 staples has a unique address with ˜6 nm resolution on the assembled structure, constituting a “pixel” on a uniquely addressable DNA nanoarray. Each array can carry up to 200 elements, ranging from organic dyes, metal nanoparticles, quantum dots, carbon nanotubes, and proteins. For a comprehensive review on conjugating DNA with nanomaterials see [18]. DNA origami-based addressable nanoarrays have been demonstrated for a wide range of applications, ranging from single nucleotide polymorphism (SNP) detection [19] to studying protein interactions [20]. For an in-depth review on applications see [21].

Large-Scale 2D DNA Nanoarrays: State of the Art

Direct Origami Placement (DOP) [22, 23] combines top-down fabrication techniques, such as electron beam lithography, with bottom-up self-assembly to accurately place and orient an array of ˜100 nm-wide DNA origami nanostructures on a micro- or macro-scale surface, as shown in FIG. 5A. Using BOP, Gopinath et al. [24] fabricated a 65,536-pixel rendering of Van Gogh's Starry Night (FIG. 5B) where each pixel is a top-down fabricated photonic crystal cavity (PCC). Each PCC is independently programmed such that a precise quantity of DNA origami triangles, equipped with fluorescent dyes will bind, thereby digitally varying the emission intensity. In 2021, Shetty et al. [25] introduced a bench-top fabrication method for DOP using self-assembled colloidal nanoparticles, thus circumventing the need for electron beam lithography. DOP enables the placement of DNA nanoarrays on a macro-scale. Yet, each triangle consists of the same staples. Thus, the true addressability of the entire array remains on the order of 200, enhanced such that the quantity, placement, and orientation of each distinct triangle is independently controlled.

In 2017, Tikhomirov et al. [26] fabricated the largest 2D uniquely addressable array to date, by a pure self-assembly approach titled fractal assembly. DNA nanoarrays spanning a total area of 0.5 μm² while containing 8,704 unique addresses/pixels were synthesized using fractal assembly. To demonstrate how each pixel is uniquely addressable, Tikhomirov et al. patterned the arrays to form nanomolecular paintings of the Mona Lisa and various other patterns (FIG. 5C, FIG. 5D). Fractal assembly approaches the scale challenge by a multi-stage strategy. First, basic DNA origami tiles are designed and self-assembled such that 2×2 tiles connect to form a larger tile. In the next step, these larger tiles are assembled together to form an even larger, 4×4 tile, and so forth. Each starting tile is precoded with pixels, by extension of the uniquely addressable staples, and the edges of the tiles are designed to accurately control the multi-stage assembly. The authors theoretically and experimentally calculate the assembly yield probability for two tiles as 0.95. Since the process has no error-correction scheme, and every error is propagated globally, total assembly yield is 0.95^{m-1}, where m is the total number of tiles. The record number of 8,704 pixels was generated in three steps using 8×8=64 tiles per pattern. Hence, the yield for the Mona Lisa pattern is 0.95⁶³=3.9%. If a pattern with a higher number of pixels/tiles would have been attempted, for example ˜40K pixels in ˜200 tiles, the yield would drop to <10⁻⁴. Thus, without further innovation, fractal assembly cannot be scaled beyond >10K pixels.

In biological systems, DNA serves as a carrier of hereditary information, facilitated by predictable and programmable base-pairing rules. The field of DNA nanotechnology takes the DNA molecule out of its original context and using the same set of rules to construct complex structures and molecular machines at the nanoscale regime. At the nanoscale, precise organization of biological and non-biological materials in 2D or 3D space holds great promise for a vast range of applications in areas such as biophysics, point-of-care diagnostics, biomolecule structure determination, drug delivery, and more. Nucleic acid scaffolds, especially DNA origami, have emerged as a promising approach, by enabling <10 nm assembly of nanomaterials such as gold particles, carbon nanotubes, and quantum dots. Two-dimensional DNA nanostructures with a plurality of uniquely addressable linkage sites (“nanopixels”) are known as DNA nanoarrays.

Expanding the size of DNA nanoarrays is desired for a variety of applications, from whole genomic sequencing at a fraction of the cost to sustainable digital information storage. Yet, due to the stochastic nature of self-assembly, DNA origami-based approaches suffer from an inherent scale limit. Top-down fabrication techniques enable nanometric precise patterning, yet single-molecule placement remains a daunting challenge. Currently, no method enables independent nanoscale manipulation of more than 10K diverse single-molecules.

The synthesis cost per base still remains a significant hurdle for the widespread adoption of DNA storage. The current cost of standard synthesis methods is approximated at $800 per Megabyte [107]. Recently, Lee et al. demonstrated a proof-of-principle enzymatic synthesis method [108] that reduces the cost per base to an order of $1 per Megabyte. Still, standard storage devices prices are multiple orders of magnitude below this threshold, with magnetic tape cost at $16 per Terabyte [109]. Even if we take into account the maintenance costs of magnetic tape, which are three orders of magnitude higher compared to DNA storage maintenance [102], still, DNA storage expenses must decrease in four to five orders of magnitude to serve as a financially competitive alternative.

As can be seen, there is a need for a low cost method of independent nanoscale manipulation of more than 10K diverse single molecules to produce uniquely addressable DNA nanoarrays.

### SUMMARY OF THE INVENTION

In one aspect of the present invention, a DNA nanoarray comprises a milliscale chip substrate; a binder bound to the milliscale chip substrate as a microscale spot having a uniform surface; and immobilized oligonucleotide sequences, each having a linker linked to the binder such that the immobilized oligonucleotide sequences form a monolayer and each of the immobilized oligonucleotide sequences having a length of at least N. N is a minimum length operative to guarantee within a statistical certainty that the immobilized oligonucleotide sequences are each unique.

In another aspect of the present invention, a method of producing a DNA nanoarray comprises providing a streptavidin-coated substrate; patterning the streptavidin-coated substrate by photolithography to produce a patterned surface having an array of microscale spots with active streptavidin binding sites; and immobilizing biotin-tagged oligonucleotides on the patterned surface by applying a solution containing the biotin-tagged oligonucleotides to the array of microscale spots; applying a buffer over the patterned surface; and washing the patterned surface in buffered saline solution. The biotin-tagged oligonucleotides each have a string of bases with a length operative to guarantee within a statistical certainty that the string of bases of the immobilized biotin-tagged oligonucleotide are each unique.

In another aspect of the present invention, a method of storing information on and retrieving information from the DNA nanoarray comprises providing the DNA nanoarray; writing bits to and/or storing spatial patterns to a subset of the nanopixels on the DNA nanoarray; and reading and/or visualizing the bits and/or the spatial patterns.

These and other features, aspects and advantages of the present invention will become better understood with reference to the following drawings, description, and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a prior art image of a spatial arrangement of a school of fish in M. C. Escher's wood engraving Depth (1955);

FIG. 1B is a prior art schematic diagram illustrating self-assembly of branched DNA molecules;

FIG. 1C is a prior art schematic view illustrating ligated DNA molecules forming interconnected rings;

FIG. 2 shows a prior art compilation of nanoscale 2D and 3D structures;

FIG. 3 is a graph illustrating prior art forecasting of costs of DNA sequencing and synthesis technologies;

FIG. 4 is a schematic diagram illustrating a DNA microarray on a prior art chip;

FIG. 5A is a prior art photomicrograph of a grid composed of DNA origami triangles;

FIG. 5B is a prior art image of Van Gogh's Starry Night reproduced utilizing DNA pixels;

FIG. 5C is a prior art the Mona Lisa reproduced utilizing DNA origami tiles;

FIG. 5D is a series of prior art atomic-force microscopy (AFM) images of fractal-assembly DNA patterns;

FIG. 6 is a schematic view of a method of DNA canvas fabrication according to an embodiment of the present invention;

FIG. 7 is an enlarged detail view thereof;

FIG. 8A is a schematic view of a prior art streptavidin-coated slide;

FIG. 8B is another schematic view thereof, illustrating molecular spacing of streptavidin;

FIG. 9 is a schematic top plan view of a circular DNA canvas according to an embodiment of the present invention;

FIG. 10A is a graph of a percentage of nodes belonging to a largest connected component in a simulated graph as a function of sequencing depth at 0.5 μm;

FIG. 10B is a graph of a percentage of nodes belonging to the largest connected component in a simulated graph as a function of sequencing depth at 1 μm;

FIG. 10C is a graph of a percentage of nodes belong to the largest connected component in a simulated graph as a function of sequencing depth at 2 μm;

FIG. 11 is a schematic view of a prior art spring model-based graph drawing;

FIG. 12A is a graphical evaluation of relative error of prior art graph drawing algorithms;

FIG. 12B is a graphical evaluation of accuracy error of prior art graph drawing algorithms;

FIG. 13 is a graph of the effect of sequencing depth on mapping accuracy;

FIG. 14 is a schematic view of the effect of sequencing depth on mapping accuracy;

FIG. 15 is a graph of the effect of reach distance on mapping accuracy;

FIG. 16 is a graph of the effect of compactness on mapping accuracy;

FIG. 17A is a schematic view of a DNA chiplet according to an embodiment of the present invention;

FIG. 17B is a schematic view of an active area of the DNA chiplet of FIG. 17A;

FIG. 17C is a schematic view of a DNA nanoarray in the active area of FIG. 17B;

FIG. 18 is a photograph of a DNA chiplet according to an embodiment of the present invention;

FIG. 19A is a schematic view of a basic fluorescence-based immobilization assay;

FIG. 19B is a schematic view of a hybridization-based immobilization assay;

FIG. 20A is a photomicrograph showing a result from a fluorescence-based immobilization assay, having strong signal-to-background fluorescence;

FIG. 20B is a photomicrograph showing another result from the fluorescence-based immobilization assay, exhibiting holes and/or islands;

FIG. 20C is a photomicrograph showing yet another result from the fluorescence-based immobilization assay, illustrating a “coffee stain” effect;

FIG. 20D is a photomicrograph showing another result from the fluorescence-based immobilization assay, having scratches;

FIG. 21 is a schematic illustrating a solid phase polymerase chain reaction (PCR) method according to an embodiment of the present invention;

FIG. 22A is a schematic view illustrating prior art microfabrication by photolithography utilizing an optical mask with a negative photoresist;

FIG. 22B is a schematic view illustrating prior art microfabrication by photolithography utilizing an optical mask with a positive photoresist;

FIG. 23A is a photomicrograph illustrating a result from an experiment in microfabrication utilizing patterning fluorophore-tagged DNA using electron beam lithography (EBL);

FIG. 23B is a photomicrograph illustrating another result from the experiment in microfabrication utilizing patterning fluorophore-tagged DNA using EBL;

FIG. 24 is a schematic view of a microfabrication process for patterning microscale spots on Streptavidin-coated surfaces according to an embodiment of the present invention;

FIG. 25A is a photomicrograph of microscale spots produced by the process of FIG. 24;

FIG. 25B is another photomicrograph of microscale spots produced by the process of FIG. 24;

FIG. 26A is a photomicrograph at 5× magnification of a microscale spot array produced without an adhesion promoter;

FIG. 26B is a photomicrograph at 10× magnification of the microscale spot array of FIG. 26A;

FIG. 26C is a photomicrograph at 5× magnification of a microscale spot array produced with an adhesion promoter;

FIG. 26D is a photomicrograph at 10× magnification of the microscale spot array of FIG. 26C;

FIG. 27 is a schematic view of a microfabrication process for patterning optically visible alignment markers on Streptavidin-coated surfaces according to an embodiment of the present invention;

FIG. 28A is a scanning electron micrograph of markers developed in resist before etching, according to the process of FIG. 27;

FIG. 28B is a scanning electron micrograph of the markers of FIG. 28A after etching and resist stripping;

FIG. 29 is a schematic view illustrating a prior art method of DNA microscopy;

FIG. 30A is a schematic view of a step of a prior art iterative proximity ligation method of DNA microscopy;

FIG. 30B is a schematic view of another step thereof;

FIG. 30C is a schematic view of another step thereof;

FIG. 31 is a schematic view of an oligonucleotide design and preparation process according to an embodiment of the present invention;

FIG. 32 is a schematic view of an on-chip enzymatic reaction method according to an embodiment of the present invention to generate proximity data encoded into DNA;

FIG. 33A is a chart exhibiting prior art data on the effect of temperature and time on Biotin-Streptavidin release;

FIG. 33B is a chart exhibiting prior art data on the effect of salt concentration on Biotin-Streptavidin release;

FIG. 34 is a chart showing the effect of temperature on the Streptavidin-Biotin bond in selected isothermal amplification buffers;

FIG. 35 is a prior art schematic of the working mechanism of a molecular beacon;

FIG. 36A is a chart showing a Quantitative Nicking Enzyme Amplification Reaction (NEAR) calibration curve;

FIG. 36B is a chart of fluorescence signal-to-background ratio for a NEAR solution in comparison to positive and negative controls;

FIG. 37A is a schematic view of a pattern and spatially located barcodes for a method of decorating a DNA canvas according to an embodiment of the present invention;

FIG. 37B is a schematic view of a conjugation step thereof;

FIG. 38 is a schematic view of a combinatorial decoration system according to an embodiment of the present invention;

FIG. 39 is a schematic view of a prior art method of DNA micro-array copying;

FIG. 40A is a schematic view illustrating a prior art method of sorting fluorescent cargo with DNA robots;

FIG. 40B is a schematic view illustrating a prior art localized DNA circuit;

FIG. 40C is a schematic view illustrating a prior art mobile DNA structure and a track designated therefor;

FIG. 40D is a schematic view illustrating a prior art DNA navigator maze; and

FIG. 41 is a schematic illustrating the relative size of prior art storage media to DNA storage media for the same amount of data.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, the term “nanopixels” refers to an ultra-dense nanoarray of uniquely addressable oligonucleotides in solid phase.

Unless indicated otherwise by context, the term “unique” indicates that a string of bases is generally not duplicated within a set of oligonucleotides.

The term “room temperature” refers to a well-known temperature range of about 15° C. to about 25° C., such as about 20-22° C.

The term “oligo” is used herein as an abbreviation of the word “oligonucleotide”.

The term “ligation events” is used herein to describe DNA copies, made by a set of co-localized enzymatic reactions, that indicating proximity information for each oligo.

Except where otherwise noted, the term “sequencing depth” is defined as the average number of times a particular oligo is represented in a collection of random raw ligation events.

The terms “primer site” and “priming site” are used interchangeably herein, and may include the term “primer binding site”.

Broadly, one embodiment of the present invention is a new type of DNA nanoarray and a method of producing the nanoarray referred to herein as DNA Canvas.

DNA CANVAS refers to a low-cost process that relies on pairwise barcode association. At the end of the process, each unique barcode is spatially located within 10 nm resolution. The immobilized barcode can later be addressed by complementary strands carrying varied molecules of interest. The association mechanism is based on co-localized enzymatic reactions. Thus, the yield error is invariant to the array size. Notably, array size is governed by sequencing cost. Overall, pairwise association enables a new generation of affordable, scalable, uniquely addressable DNA nanoarrays.

DNA Canvas, the result of the fabrication process, is a milliscale chip that contains a microscale spot composed of millions of uniquely addressable DNA nanopixels. Alongside a physical chip is a digital file that maps the sequence of each nanopixel to a 2D coordinate with nanoscale precision. DNA Canvas can be used for a wide range of applications that require precise nanometric positioning of molecular elements in 2D space, at a low cost.

The present subject matter blurs the lines between engineering and biology to enable accessible prototyping for a wide range of nanoscale applications using nature's machinery. DNA Canvas offers a few significant gains over state-of-the-art methods in DNA nanoarray fabrication. First, the sheer number of pixels and addressability of the array are potentially far greater compared to any current method. Self-assembly approaches such as fractal assembly suffer from an exponential relationship between the number of pixels and yield [26], due to inherent errors propagating globally. Meaning, any self-assembly mistake between two tiles prevents larger tiles in the assembly tree from being assembled correctly. Like any biological process, DNA Canvas must suffer from a certain error. However, since the enzymatic reactions are co-localized on a surface, errors strictly affect a fixed size neighborhood and do not propagate to other parts of the array. This simple notion implies there is no assembly scale limit on DNA Canvas size. As will be described infra, the scale laws governing DNA Canvas are directly tied to the costs of DNA sequencing, which have decreased by six orders of magnitude in the last two decades [7]. Hybrid methods such as DOP do not suffer from exponential decays in yield. However, while these techniques can manipulate relative placement, orientation, and quantity of DNA nanostructures, direct control over a diverse single-molecule library is still not in reach. DNA Canvas allows for specific molecules to be tagged with unique barcodes. Assuming the number of hybridized molecules is large enough (by the law of mass action), we can directly place molecules of interest in complex nanometric precise patterns. DNA origami-based nanoarrays have been used as nanofabrication assembly platforms [6, 4, 21]. However, the nanostructures are synthesized in solution and are typically deposited on a surface, leading to spatial stochasticity. DNA Canvas builds upon the bottom-up nanofabrication techniques developed for DNA origami, while enabling single-structure assembly at a fixed location on-chip, surrounded by visible alignment markers, enabling a wide range of applications that require external interface with the assembled nanostructure.

Previous work has been done to reconstruct spatial organization strictly based on local neighborhood data, within the context of DNA. Glaser et al. [29] presented puzzle imaging, where spatially disordered samples are combined into a high-resolution image based on local properties. Specifically, local information can be generated by a chemical process, e.g., DNA polymerase encoding a unique string of DNA. Recently, Hoffecker et al. [30] introduced Polony Adjacency Reconstruction for Spatial Inference and Topology (PARSIFT), a computational framework designed for decoding pairwise associations between adjacent DNA polonies into images. Distinguishably, these methods operate without a priori knowledge to reconstruct an unfamiliar topological map. DNA Canvas is distinct since the topology is known a priori—a mono-layer grid.

Barcode Uniqueness

Each oligonucleotide (oligo) contains a barcode—a unique string of bases (e.g., ATGTACCA . . . ) of predetermined length. We assume each barcode is unique on the DNA Canvas. Experimentally, this assumption is enforced by statistical means. Traditionally, oligonucleotides are synthesized base by base, in a linear series of chemical reactions. A barcode is created when at a specific step, multiple bases are used in the chemical reaction. The random choice is represented by the letter N. For example, synthesizing the sequence ATNCG will result in four different oligo sequences: ATACG, ATTCG, ATCCG, ATGCG. Similarly, synthesizing the sequence ATNNCG will result in 16 (=4²) different sequences. A barcode of length k has 4^{k} distinct possibilities. Denote d as the diameter of a circular DNA Canvas (in nanometers). It follows that there are

$$m = \frac{π \frac{{d}^{2}}{2}}{{2.5}^{2}} = \frac{π {d}^{2}}{25}$$

binding sites. The probability, by counting, of having a non-unique barcode is:

P (non-unique barcode, k, d,

$$m = \frac{π {d}^{2}}{25} ) = \frac{( \begin{matrix}{4}^{k} \\ m\end{matrix} ) m !}{{4}^{k \cdot m}}$$

For example, a circular DNA Canvas of diameter d=5 μm contains m=π·10⁶ binding sites. Given a barcode length of k=14, we approximate an upper-bound on the probability of having a non-unique barcode, by calculating the product of the largest 200,000 factors:

P (non-unique barcode, k=14, d=5000,

$$m = π \cdot {10}^{6} ) = \frac{( \begin{matrix}{4}^{14} \\ π \cdot {10}^{6}\end{matrix} ) m !}{{4}^{14 \cdot π \cdot {10}^{6}}} \leq {10}^{- 32}$$

Essentially, the relationship between k and m is logarithmic (see Proof “Barcode Length vs. Number of Binding Sites”, below), which implies that a larger DNA Canvas requires synthesis of oligos containing only slightly longer barcodes to validate the uniqueness assumption. The Barcode Length vs. Number of Binding Sites Proof gives an exact formula for choosing k given m.

Global Mapping from Pairwise Measurements

In essence, fabricating a DNA Canvas is fairly simple: oligos are synthesized with a unique barcode and tagged with a ligand molecule (e.g., Biotin). A highly concentrated solution of tagged oligos is suspended over a coated surface that binds the ligand molecule (e.g., Streptavidin-coated slide). After a short time, the surface is washed, such that only oligos that had physically bound to the surface remain. Thus, we now have a surface with a plethora of oligos, each with a unique address and location. Alas, both the barcode and the spatial location for each oligo are unknown. Algorithms that can recover these unknowns with high accuracy using available information are described herein. We aim to obtain a mapping such that each barcode (a string of bases of a predetermined length, e.g., ATGAAT . . . ) is mapped to a spatial two-dimensional coordinate (e.g., 55,100). The input to the problem is local, pairwise measurements.

As will be described experimentally, we perform a set of carefully designed enzymatic reactions—ligation, amplification, and restriction. In the ligation step, each pair of adjacent oligos is connected. Next, copies of connected oligos are synthesized. Each copy contains two barcodes—one for each oligo in the pair. Then, the two oligos are disconnected. The last step is critical to have independent iterations. In each cycle, a different pair of oligos is ligated (each oligo can have multiple neighbors). The output of this process is the DNA copies, where each copy containing two barcodes, that must be adjacent on the surface. Last, we perform next-generation sequencing. Sequencing transforms physical DNA copies into digital information. In effect, we get a long list of DNA sequences, each with two unique barcodes.

This computational problem of global mapping from local proximities is also known as the Molecule Problem [33] or the Graph Realization Problem [34] and has been applied in a variety of settings from wireless sensor networks to structural biology. The present disclosure enables one to leverage an important topological assumption—the oligos are uniformly distributed in 2D space. This assumption gives rise to a set of algorithms that can solve the problem with high accuracy, short time, and limited computational resources.

Graph Representation

The DNA Canvas computational problem can be treated as a graph. Each barcode is a node. An edge exists between two nodes if a pair of barcodes appears in the list of measurements. Edges and nodes are unweighted. The goal is to find a coordinate for each node—an embedding of the graph in 2D space. In our setting, each edge represents a ligation and amplification event between a 5′ oligo to a 3′ oligo. These enzymatic reactions can only happen if the two oligos can reach each other. Furthermore, we assume that the oligos are uniformly distributed on the surfaces, hence the embedding of nodes should be uniformly distributed in 2D space. The number of edges is dictated by the number of ligation rounds and sequencing depth.

Parameter Space

There are a few key parameters in DNA Canvas design. Each of the parameters acts as a “knob” that can be fine-tuned to optimize for resolution.

Methods

All experiments in this disclosure were written in Python 3. Graph data structures and algorithms (Fruchterman and Reingold [37], Scalable Force Directed Placement—SFDP [40]) were implemented by the graph-tool [46] library, a highly efficient Python module manipulation and statistical analysis of graphs. The high-dimensional embedding method [42] was implemented by the author, with the support of NumPy [47] and scikit-learn [48] libraries. Figures were generated using the Matplotlib [49] and seaborn [50] libraries. All experiments were executed on a single- or multi-core Intel Core i7-8550U CPU @ 1.80 GHz.

Fruchterman and Reingold was executed for 100 iterations in grid mode, where repulsive forces only act on a local neighborhood. High-dimensional embedding was implemented with a random sample of 1% of the nodes act as pivots. SFDP was run using an adaptive cooling scheme.

Since 2007, Next-Generation Sequencing (NGS) costs had plummeted by six orders of magnitude, with a current price tag of approximately $0.01 per 10⁶ bases [7]. DNA Canvas relies on NGS of ligation events between immobilized oligonucleotides (oligos). Each oligo serves as a nanopixel, with a 2D coordinate and address. Importantly, to achieve medium-to-high resolution we must sequence an ample amount of the entire DNA Canvas surface. Briefly, we need to sequence at least four times (4×) more ligation events than the number pixels to be close to 10 nm accuracy. The size of each ligation event is twice the length of the oligos. Meaning, to fabricate a DNA Canvas composed of N nanopixels (each k bases long), we have to sequence at least 8 kN bases, which currently costs $8·10⁻⁸ kN. For example, a DNA Canvas with 1M nanopixels (k=50) could cost merely $4.

The enzymatic reactions that generate the pool of ligation events all happen in a liquid volume on the surface where the oligos are immobilized. Assuming the 2.5 nm S-B model described herein, a 1M nanopixels circular Canvas would span a spot of diameter

$$d = \frac{5}{\sqrt{π}} \approx 2.8 µm .$$

For reference, a commercially available lab pipette can handle droplets of volume 0.1 microliter, while state-of-the-art inkjet printers can dispense droplets of 1-10 picoliter. Due to the cubic relationship between the diameter and the volume of a droplet, the volume of half a droplet of diameter 2.8 μm is 16 fL—two orders of magnitude below the capabilities of advanced inkjet printers.

Thus, we are left with a few alternatives. One, we could aim to fabricate a Terascale DNA Canvas that spans >1 mm, which would imply an exorbitant sequencing cost (>$1M). Nonetheless, as part of the roadmap, we will chart an alternative path to a Terascale Canvas at a reasonable cost. Alternatively, advanced methods exist for direct femtoliter [51] or even attoliter [52] droplet dispensing, yet these require complex setups. Recently, advancements in nanofabrication and photolithography had led to a wide adoption across academia and industry. This set of tools and techniques serves as an ideal solution for the problem of microscale DNA spots fabrication, in a matter that is both affordable, scalable, and reproducible.

DNA Immobilization

Glass slides have been widely used for DNA immobilization for decades to fabricate DNA chips for a wide range of applications [53]. Glass serves as an ideal substrate due to excellent optical properties and minimal compatibility issues with most organic solvents [54]. Numerous techniques have been applied to effectively immobilize DNA onto glass surfaces [55]. Overall, methods usually require some chemical modification of the surface as well as tagging the oligonucleotide (oligo) with a linker upon synthesis. Roughly, methods can be divided by the linking chemistry: covalent and non-covalent bonds.

Covalent linking methods include Epoxy or Aldehyde modified surfaces combined with Amine-tagged oligos, or alternatively gold (Au) modified surfaces with thiol-tagged oligos. These techniques offer excellent stability, high binding strength, and long-term usage, yet can suffer from problems such as crowding effect or island forming [55].

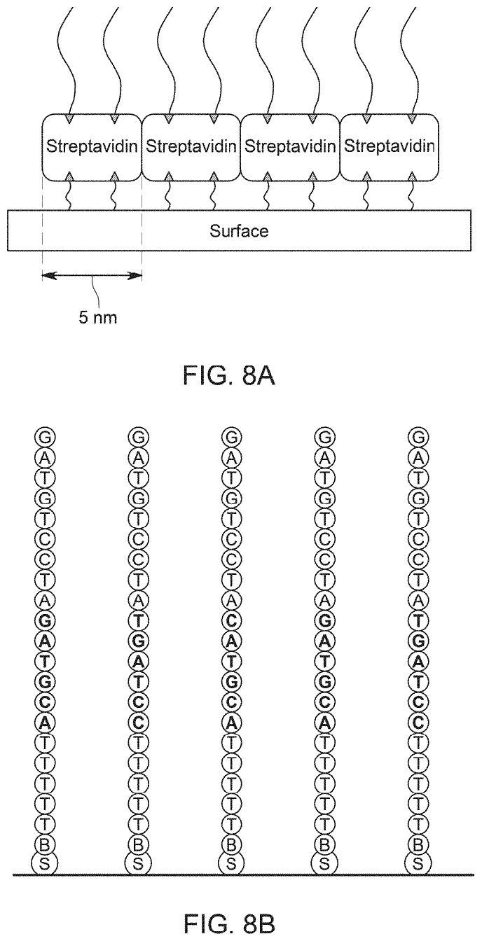

Streptavidin-Biotin binding is the most common non-covalent based approach and is used in a wide variety of scenarios [56]. Streptavidin is a tetramer protein that exhibits extremely high affinity (Kₐ=10¹⁵M⁻¹), nearly covalent, to ligand protein Biotin [31]. Generally, the surface is first modified with a layer of Biotin, and then Streptavidin is added (FIG. 8A).

Streptavidin-Biotin-based DNA immobilization is an attractive option for DNA Canvas due to the simple and quick protocol as well as the predicted surface density that is governed by the size of the Streptavidin molecule (5 nm [32]). Determining the area of a single nanopixel is important for two main reasons: first, it is a key assumption for the computational model in charge of localization; second, it allows for efficient hybridization. In contrast to prior art DNA microarrays, where every spot is a colony of oligos with the same sequence, each barcode in our DNA nanoarray appears on a single molecule. Therefore, we need to assert that each oligo has adequate spacing for a complementary probe to bind. Nonetheless, covalent methods still serve as an excellent alternative, especially for production-grade devices, thanks to their long-term stability. The present disclosure also envisions use of Epoxy-Amine linking.

Chiplet Form Factor

Generally, coated glass surfaces are manufactured either in a form factor of a microscope slide (25×75 mm) or as a wafer (4 or 6 inches). The fabrication process of a DNA Canvas entails three steps: immobilization, enzymatic reactions, and computational localization. The first step can be done by suspending a nonreactive droplet over a coated slide or wafer for a short period of time (15-60 minutes). However, the second step requires iterative cycles where three different reaction solutions are suspended over the surface.

Enzymatic reactions on a surface (“chip”) are a wide and prominent research area, also known as Lab-On-Chip [57]. A relevant technique to our setting is Solid Phase PCR (SP-PCR) [58], where DNA primers are immobilized to a surface and the amplification happens on-chip. Every reaction has an aqueous media, a carefully designed mixture of salts, buffer, enzymes, and nucleotides aimed to optimize the reaction efficiency. Therefore, the solution cannot be allowed to dehydrate over time. Commonly, SP-PCR and other on-chip reactions use either seal chambers [59], oil encapsulation [60] or microfluidics devices [61] to guarantee the reaction conditions stay stable. While seal chambers or encapsulation in mineral oil are both simple and accessible methods, our protocol requires frequent changes to the reaction solution. Furthermore, we need to store the solution for downstream sequencing. A custom microfluidic device could serve as an excellent solution, yet we opted for a simpler approach.

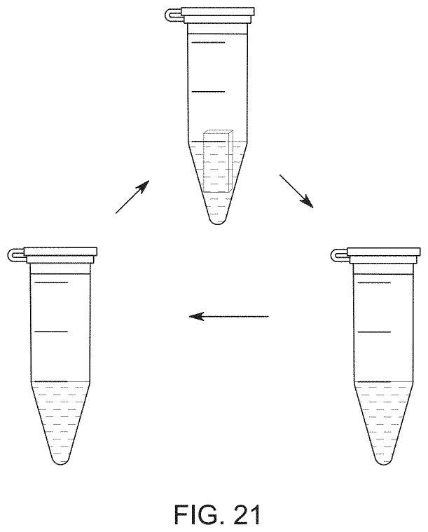

Instead of suspending an aqueous media on the surface of a slide, we dice the slide into small pieces (chiplets) that can be suspended inside an aqueous volume. Specifically, we dice the coated slide to chiplets that can fit exactly into the bottom of a commercially available PCR tube (3×6 mm), a common lab instrument that can host reactions in volumes of 10-50 μl. Three 50 μl tubes are prepared containing the needed buffers and enzymes, each tube having a specific reaction condition, and the chiplet is cycled between the tubes, as shown in FIG. 21. The tube shown has an upper diameter of 5-6 mm.

The present disclosure envisions upscaling the fabrication process per chiplet, in terms of time and costs. EBL may, in some cases, be used for nanoscale writing onto DNA Canvas (e.g., DNA Information Storage), although for fabrication purposes, the EBL time of 15-60 minutes per 1 cm² does not scale well. The direct-write photolithography tool, MLA-150, proved to be especially useful for prototyping, with an exposure time of ˜2 minutes per 0.5×0.5 inch². The evident next step beyond prototyping is mask aligner-based fabrication. Mask aligners are tools to use a predetermined optical mask and can transfer a pattern over to a large area of resist at a fixed constant time. Thus, using a commercially available mask aligner, a 6-inch wafer that contains ˜1000 chiplets can be fabricated in 30 seconds.

Last, while we chose to focus on Streptavidin-Biotin chemistry, the present disclosure further envisions various linking methods, specifically covalent ones.

At present, DNA microscopy had been experimentally applied only a handful of times. In 2017, Schaus et al. [67] were the first to validate this approach, by immobilizing seven synthetic probes on a DNA Origami substrate with predetermined distances between each probe. Using Auto-cycling Proximity Recording (APR) method, immobilized hairpin probes designed with palindromic domains are interchangeably hybridized and amplified. The main limitation for APR is the probe design, which requires synthesis of a unique barcode and its complement on a single strand, such that mixed bases synthesis is irrelevant, making synthesis cost linear in the number of probes. In 2019, Weinstein et al. [28] applied DNA microscopy at scale for the first time to visualize the spatial organization of mRNA molecules (10⁵-10⁶) inside a cell, strictly based on DNA interactions. Each ligation and amplification event with a Unique-End-Identifier (UEI) can diffuse through the cell, such that the output of the computational model is a diffusion map for each unique probe identifier or unique molecular identifier (UMI). In 2020, Gopalkrishnan et al. [70] showed how hundreds of probes immobilized on a DNA Origami surface can be precisely localized at 2 nm precision, using a molecular ruler approach. Using a molecular ruler, each ligation event holds information on the physical distance between the two immobilized probes. Further in 2020, Ambrosetti et al. [66] experimentally demonstrated how DNA microscopy can map membrane protein nanoenvironments, using a technique called NanoDeep where a synthetic comb made of oligos is built to scan a membrane. Amplification and sequencing can reveal adjacency information of the membrane proteins and therefore a global map.

We build upon the work described above by applying DNA microscopy in a novel scenario—on-chip. We fabricate a chiplet able to store vast amounts of compact unique oligos (>10⁶). This particular setting allows us to scale beyond previous attempts. Our goal is not topological imaging, as we already know the surface is flat and packed with binding molecules. Instead, our aim is precision mapping between a barcode and a spatial location, in order to generate a device useful for downstream applications. Here, we present Chip-based Iterative Proximity Ligation and Extension (ChIPLEx), an on-chip enzymatic reaction method to generate proximity data encoded into DNA.

Sensitivity to Elevated Temperatures

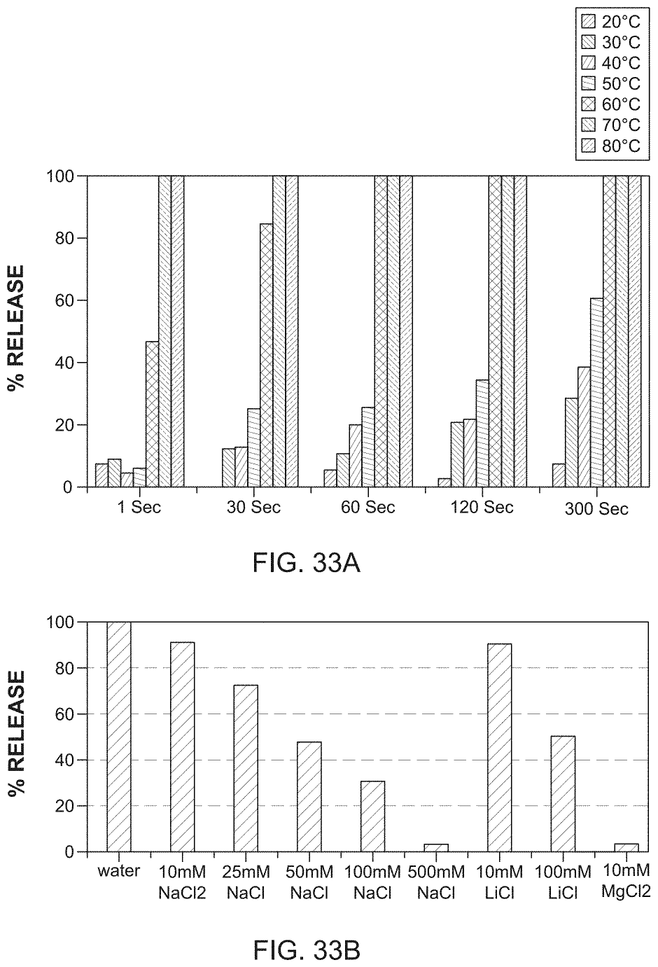

The bond between Streptavidin and Biotin has long been regarded as the strongest, non-covalent, biological interaction known in nature (Kₐ≈10¹⁵M⁻¹) [31]. The bond forms very rapidly and is stable in wide ranges of pH and temperatures. [74, 75]. Nonetheless, Holmberg et al. [71] had rigorously studied the conditions under which Streptavidin-Biotin bonds can be reversibly broken (FIGS. 33A and 33B). As can be seen in FIG. 33B, suspension in water at 70 C for just one second results in a 100% release of Biotin-tagged molecules. For high concentrations of salts, the percentage drops to less than 5%. Since Nicking Enzyme Amplification Reaction (NEAR) reaction is typically run at elevated temperatures of 55-65° C., we set to find which buffer and salt conditions are compatible to minimize Biotin-tagged oligos release.

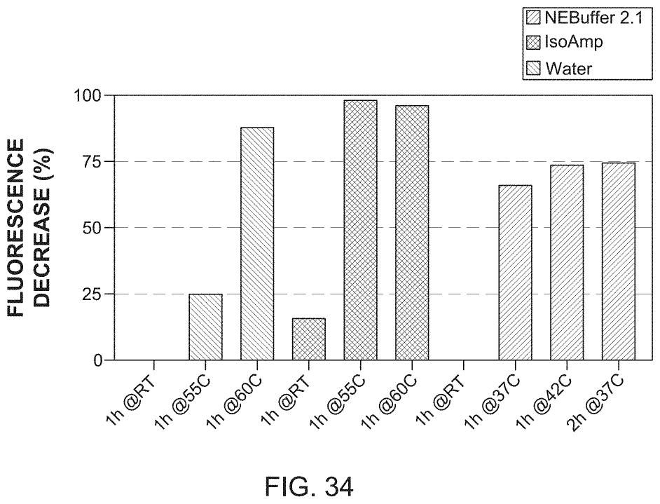

FIG. 34 presents the results of our investigation. We tested various buffers that support common DNA polymerases with strand displacement activity (Bst, Bsu, Klenow), as well as water. Bst Polymerase is the original polymerase used in the popular isothermal LAMP reaction [76], as well as NEAR [77]. The buffer of choice for Bst 2.0 Polymerase is Isothermal Amplification Buffer (IsoAmp, New England Biolabs). The recommended temperature range for Bst Polymerase is 50-65 C. NEBuffer 2.1 (New England Biolabs) is a known buffer that supports both Bsu Polymerase and Klenow Large fragment. Bsu Polymerase preferred temperature range is 37-45 C, while Klenow is active at room temperature. The experiment was conducted as follows: Biotin-tagged oligos, attached with a fluorescent dye (Cy3), are immobilized to a chiplet. The chiplet is observed under a fluorescence microscope to validate the initial immobilization was successful. Then, the chiplet is suspended for one hour inside a PCR tube containing a certain buffer at a precise temperature (using a Thermocycler for temperature control). Simultaneously, another DNA chiplet is suspended in water at room temperature. The results show fluorescence intensity decrease relative to the chiplet that remained at room temperature for one hour.

The results demonstrate that Biotin-Streptavidin bond is sensitive to elevated temperatures for relevant buffers. Consequently, we design the reaction and enzymes to be optimal at room temperature.

Steric Hindrance

A design constraint when running enzymatic reactions on-chip is steric hindrance due to surface proximity. Therefore, it is a common practice to add a “spacer” between the attachment molecule and the enzyme's recognition site for on-chip reactions [78, 79]. Spacers can be either nucleic acid sequences or molecules such as Polyethyleneglycol (PEG) that are synthesized within the oligonucleotide. Spacers have been shown to increase hybridization yield [80] and enzymatic efficiency [81].

We devise an assay to validate the optimal spacer length for DNA Canvas. We synthesize a Biotin-tagged oligo, composed of a spacer, a priming site, and a ˜20 bases sequence simulating the barcode region, yet is limited to nucleotides A/C/T. At the 3′ end of the oligo, there is either a Guanine (G) or a Cytosine (C). The protocol (on-chip enzymatic fluorescent labeling) starts by immobilizing a pool of oligos to the surface. Afterward, an enzymatic extension reaction is carried on-chip by a DNA Polymerase. The building blocks for every extension reaction are nucleoside triphosphates: dATP, dCTP, dGTP and dTTP. DNA Polymerase is able to synthesize these blocks onto the coding strand using base-pairing rules. If the template sequence contains the nucleic acid Guanine (G), dCTP will be attached, and so forth. The assay works by supplying ample amounts of dATP, dGTP, dTTP yet no dCTP. Instead, a dCTP-Cy3 (Jena Biosciences) is used, which is a modified dCTP attached with a fluorescent dye (Cy3). Thus, if the surface has oligos that have Guanine (G) at their 3′ end, we expect to observe fluorescence. If there is no Guanine in the template strand, no significant fluorescent signal is expected after the surface is washed.

Using this assay, a spacer strategy for 3′ and 5′ Biotin-tagged oligos has been validated. For 3′ Biotin tagged oligos, a spacer composed of a single Hexaethylenglycol (HEG, sometimes referred to as PEGS) along with nine Thymines (polyT₉) was sufficient to observe a strong fluorescent signal strictly for oligos containing Guanine. Moreover, for 5′ Biotin tagged oligos, a spacer composed of two HEGs and three Thymines was sufficient.

Computational Sequence Design

NUPack [82] is a comprehensive computational framework for analysis of nucleic acid sequences over complex scenarios. Using the recently released NUPack Python module, we computationally designed the sequence to minimize the probability for unwanted interactions between the oligonucleotides and the primers. Table 1, below, presents an example DNA sequence with barcode length 14 for ChIPLEx. In bold are endonuclease recognition sites. N represents a random (mixed) base.

TABLE 1
OPTIMIZED SEQUENCE

| Role | Sequence (5′ to 3′) |
|---|---|
| Attachment | 5′-Biotin |
| Spacer | (TEG)-T-(HEG)-TT-(HEG) |
| Rev. Priming Site | CGCACGCATCCAGAC |
| Barcode | NNNNttNNNttNNNttNNNN |
| Restriction Site | GGGCCC |
| Barcode | NNNNttNNNttNNNttNNNN |
| Fwd. Nicking Priming | CCTCCACCGAcctcagcACTACACC |
| Site | |
| Spacer | (HEG)-TTTTTTTTT |
| Attachment | 3′-Biotin |

Notably, the endonucleases recognition sites are 6+ bases long. To prevent the endonucleases from cleaving or nicking the random barcode region, the barcode sequence was interlaced with two Thymines to prevent the 6+ bases recognition sequence (that does not contain TT) from ever appearing. The spacer regions are designed as result. The reverse priming site length is 15 bases, with estimated melting temperature (Tₘ) of 53.9° C. The forward priming site is 25 bases long and contains a nicking site for Nb.BbvCl. Post nicking, the resulting priming site is 15 bases long with a melting temperature (Tₘ) of 53.4° C.

Real-Time Quantitative Near Using Molecular Beacons

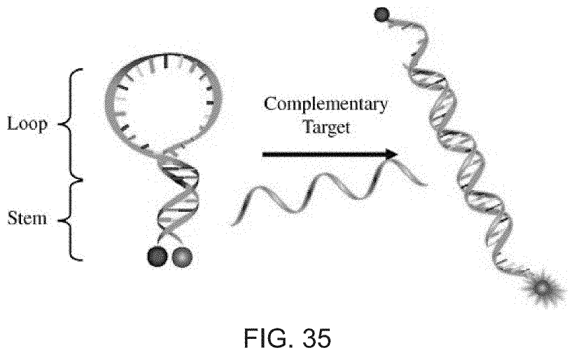

Molecular Beacons (MB) are specifically designed DNA hairpin structures that are widely used as sensitive fluorescent probes [83, 84]. As can be seen in FIG. 35, the mechanism underlying MB relies on a fluorophore and a quencher. When in close proximity, the fluorophore is strongly suppressed. When the target sequence hybridizes with the MB loop domain, the stem helix is forced to open up and the fluorescence is restored due to the spatial separation of the fluorophore from the quencher.

MB have been widely employed as a quantitative assay for PCR [85] and LAMP [86]. Here, we leverage MB to quantify NEAR in real-time. We synthesize a DNA probe using the same sequence design mentioned above yet fixing the barcode region to a predetermined sequence. The complementary sequence to the fixed barcode acts as the MB's target. For all MB experiments, we follow Vet and Marras [85] strategy of applying MB in real-time PCR assays. Particularly, we use the metric signal-to-background ratio (S/B). S/B is calculated by measuring three quantities—the fluorescence intensity of the buffer (without MB), denoted F_{buffer}. The fluorescent intensity of a solution containing MB without the target, denoted F_{closed}. The fluorescent intensity of the sample of interest, containing the target and MB, denoted Fₒₚₑₙ.

S/B is defined as:

$$Signal - to - Background Ratio = S / B = \frac{{F}_{open} - {F}_{buffer}}{{F}_{closed} - {F}_{buffer}}$$

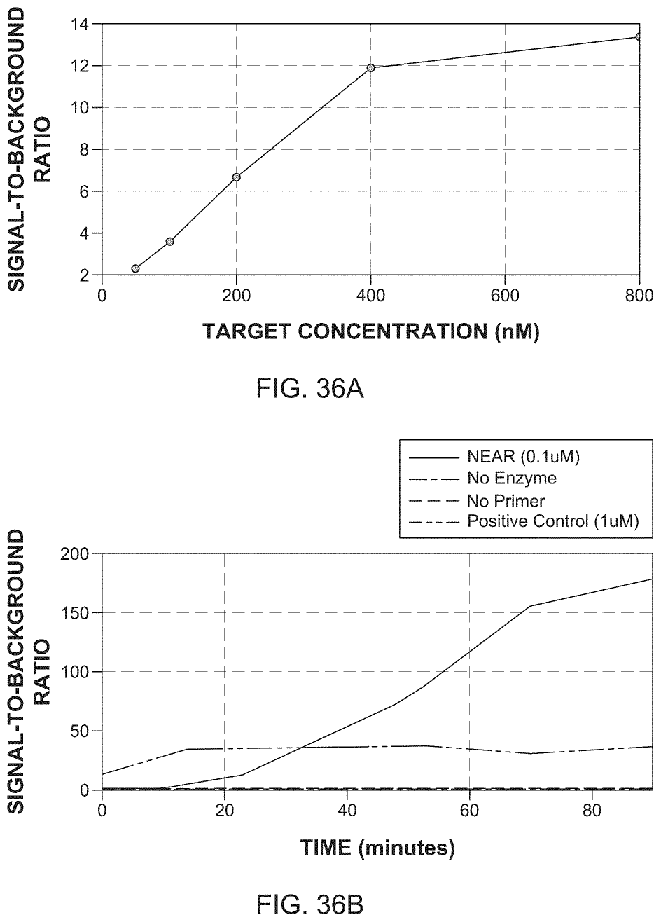

First, we generate a calibration curve for different known concentrations of the target oligo. As can be seen in FIG. 36A, we achieve a linear detection curve between 50 nM to 0.4 μM of target oligo, using 0.2 μM MB.

Next, we perform NEAR starting with a template oligo concentration of 0.1 μM. The template oligo does not contain the target sequence, but a nicking priming site and the fixed barcode region. As a positive control, we set up a reaction containing the target oligo at 1 μM. For negative control, we set up two reactions: one lacks the enzymes (DNA Polymerase and nicking endonuclease) and the second lacks the primer. All reactions are incubated at room temperature and sampled every 10 minutes using a fluorescent microplate reader. The results are shown in FIG. 36B. As expected, positive control exhibits a nearly constant S/B=40 (notably, it takes ˜10 minutes for the MB to bind to all targets), while the negative control exhibits no S/B. For the actual reaction, we see a linear amplification curve, that passes the positive control after ˜35 minutes. Therefore, we can roughly estimate NEAR kinetics under this setup (see “Nicking Enzyme Amplification Reaction” protocol described below)—10× amplification in ˜35 minutes.

The present disclosure envisions performing NEAR on-chip. To validate the results of such a reaction, we can either use the Molecular Beacon strategy (Real-time Quantitative NEAR using Molecular Beacons, supra) or simply by gel electrophoresis. The present disclosure envisions attaining a deeper understanding of the system kinetics by analyzing NEAR amplification rates as a function of parameter space. Last, combining the three reactions: ligation, extension, and cleavage into a single temperature-controlled reaction, similarly to PCR, would allow to truly scale the number of ligation events per oligo, thus minimizing the RMSD error.

DNA Sequencing and Fluorescence Sampling Methods

All DNA sequences, including the Molecular Beacon, were analyzed and optimized using the NUPack® [82] computational framework. All protocols for the biological experiments are described in Appendix B. Real-time fluorescence sampling was done using Synergy H1 Microplate Reader (Biotek®), using fluorescence mode with excitation/emission of 488/510 nm, corresponding to fluorophore 6-FAM (6-carboxyfluorescein).

Patterning/Decoration

The organization of nanomaterials such as gold nanoparticles, quantum dots, and carbon nanotubes with nanoscale precision is one of the central challenges in nanotechnology. A wide variety of nanomaterials can interface with DNA through a range of conjugation techniques, which have been well summarized in a previous review by Samanta and Medintz [18]. Currently, the leading DNA-based self-assembly method for nanoparticle manipulation is DNA Origami, as previously reviewed in [21, 4, 87]. DNA Canvas provides an attractive, complementary approach for nanomaterials placement. DNA Canvas can leverage techniques originally developed for DNA Origami, where carbon nanotubes or gold particles are modified with nucleic acids to enable hybridization at predetermined locations on the DNA-based scaffold. DNA Canvas acts as a different kind of DNA-based scaffold, that differs in phase (solid vs. liquid) and scale (>1M vs. 10K) compared to state-of-the-art DNA Origami.

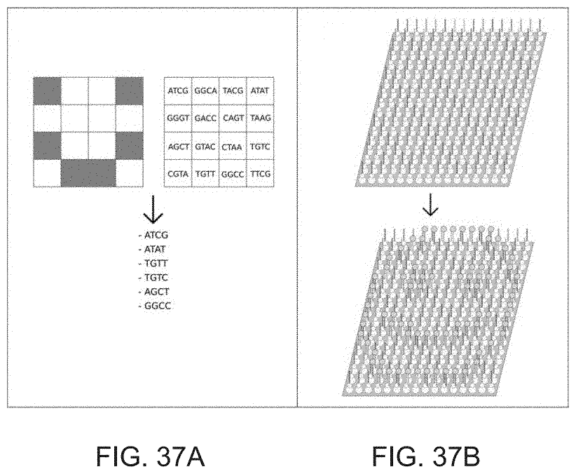

Prior to patterning nanomaterials, the first step would be a purely DNA-based proof-of-concept. We will design and synthesize DNA strands that form secondary structures such as hairpins that can later be imaged by Atomic Force Microscopy (AFM), a key tool for nanoscale imaging of DNA structures [88]. Specifically, given the list of spatial locations and barcodes, we can digitally transform any pattern to a list of complementary sequences (FIG. 37A). Then, we synthesize these complementary sequences along with a sequence that forms a hairpin structure to create a binary topological map, imaged by AFM.

Combinatorial Approach to Low-Cost Decoration

Decorating a DNA Canvas using complementary strands requires nucleic acid synthesis. Explicitly, to create a pattern composed of 100K pixels on top of a 1M DNA Canvas, it seems we would have to synthesize 100K unique oligonucleotides. Current DNA synthesis costs for short strands are on the order of $0.15 per base (Genewiz), not including complex modifications such as linker molecules. Thus, trivial patterning of 100K pixels with barcode length 14 per pixel pattern would cost at least $200K.

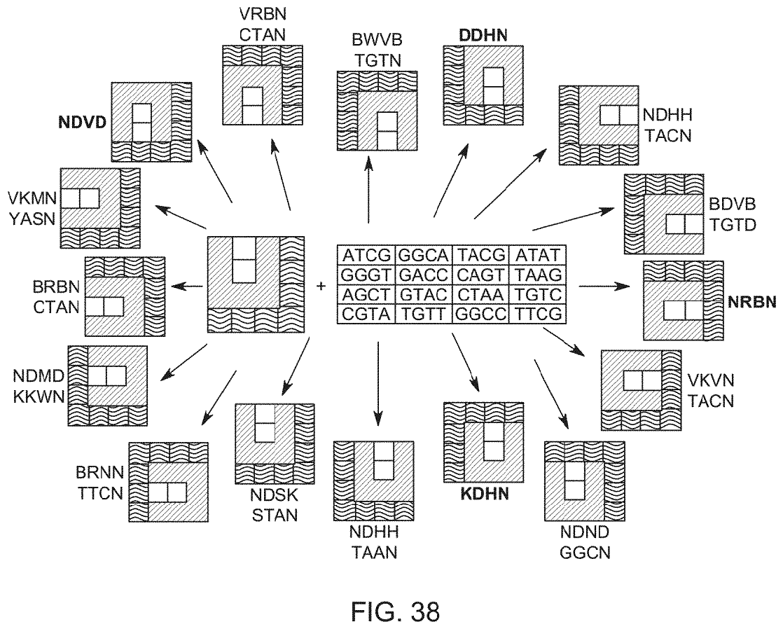

For this reason, we introduce an approach to dramatically reduce decoration costs. Oligonucleotides can be synthesized with mixed bases, also known as wobbles or randomized bases, without incurring extra costs. Using a degenerate bases notation, we can specify any subset of {A,C,G,T}, for example, R is NG; B is C/G/T; N is any base, etc. For the full nomenclature see [9], the disclosure of which is incorporated by reference in its entirety. For any library of ACGT sequences, we can reduce the number of synthesized oligos using degenerated oligo synthesis, such that the tube containing the synthesis results contains the same sequence library. Compression from ACGT to mixed bases is akin to Karnaugh maps [89], a method for simplifying a Boolean expression to a minimum number of logic gates in electrical circuit design. Similar to Karnaugh maps, defining “Don't Cares” (pixels that are irrelevant whether hybridized or not, or barcodes that do not appear on the Canvas) can greatly reduce the library size. Furthermore, for many applications the absolute location and orientation on the chiplet are insignificant. As a result, there is a vast conformations space to encode the same pattern, up to translations, rotations, or symmetrical transformations. Thus, the algorithm for computing a low-cost decoration synthesis library will search over the conformation space for a global minimal cost, dictated by the number of mixed bases sequences. This problem can be reformulated as the NP-complete, set cover problem, although, approximation methods exist, especially since our input domain (four-letter options, barcode length k) is relatively limited compared to classic set cover. FIG. 38 demonstrates a toy example of this concept. The input is a specific pattern and a mapping between pixels and barcodes. All distinct conformations are visualized by translating and rotating the pattern. For each conformation, the minimal mixed bases sequence is computed by exhaustive search (barcode length is 4). A few conformations result in a single mixed base sequence, instead of the trivial seven sequences needed to decorate the pattern.

From DNA Canvas to DNA Blackboard

Reusability is a crucial step on the path to accessible nanoscale patterning. The melting temperature (Tₘ) of double-stranded DNA is defined as the temperature for which half of the strands are in the random coil or single-stranded state. Therefore, a pattern can be removed from the surface post hybridization simply by controlled heating. For barcodes of length 14, the melting temperature can vary between 37 C to 80 C, depending on the ratio of C/G vs. NT. A computational tool can be built such that for any given conformation, the Tₘ to melt the pattern off the surface is calculated. This approach also opens an opportunity for nanometamaterials patterning, meaning, patterns that transform as a response to temperature.

Streptavidin-Biotin bonds are sensitive to elevated temperatures. This property could be mitigated by a well-designed mixture of buffer and salts. Moreover, switching to a different linking chemistry, especially one based on covalent bonds to the surface, would function as a sustainable solution allowing for reusability with minimal degradation.

Low Cost Replication

The cost per pixel of a DNA Canvas chip is dictated by next-generation costs, as elaborated supra. Next-generation sequencing (NGS) costs have been dropping exponentially since 2007, with a current price of $0.01 per 10⁶ bases [7]. Each pixel on a DNA Canvas requires sequencing of at least four distinct ligation events, where each event contains two barcodes of length k (along with some fixed length components such as primers and enzyme recognition sites). Briefly, the relationship between the number of nanopixels and the sequencing cost is linear. For example, a 1M DNA Canvas with barcode length 14 would cost merely $4, while a 1G DNA Canvas with barcode length 20 would cost ˜$4,000 to fabricate.

Why do we need to scale beyond 1G nanopixels? Some applications require milliscale or larger chips that support accurate nanoscale immobilization by DNA. Since every nanopixel must have at least four neighbors within its reach distance for the computational step to succeed, milliscale chips are bound to have >10¹² nanopixels. Notice that even if the application requires only a limited number of nanopixels across the milliscale chip, the fabrication process requires sequencing of the entire surface.

How do we scale beyond 1G nanopixels, at a reasonable cost? First, given the trends of NGS costs over the last few decades, perhaps we need not worry, as we will continue to see significant price drops per base. Alternatively, an independent approach would be to produce an expensive, main copy of a DNA Canvas, along with low-cost replicates. Ideally, each of the replicates would contain the same barcodes at the exact same locations, thus sparing the need for the costly sequencing step. In 2019, Krämer et al. [90] developed a copy-paste technique for DNA microarrays, using polydimethylsiloxane (PDMS) cavity chips that hybridize and extend off the surface of an existing DNA microarray. The PDMS chip can later be transformed to a blank slide to produce a replicate. This disclosure envisions scaling down the copy technique to the single-molecule level while minimizing the copy error rate.

Applications

DNA Canvas is a versatile platform. By exploring the design space and focusing on a low-cost fabrication scheme, we hope to enable a plethora of applications and use-cases across academia and industry. Nonetheless, on the roadmap to wide adoption lies the “killer app” milestone, (at least) one impactful application enabled by the novel DNA nanotechnology described here.

Whole Genome Resequencing

Now that reference genome sequences for many organisms (Homo sapiens included) are available, research of genomic variation and its biological consequence with regards to a reference genome, known as genome resequencing, is an active field within academia and industry. Commonly, DNA microarrays, such as the Affymetrix GeneChip® [91], are fabricated with a predetermined selection of probes, for example, single nucleotide polymorphisms (SNPs) in genes of interest in humans.

The whole human genome is 6.4 billion bases. We can fabricate DNA chips with billions of unique nanoscale features, allowing for whole human genome coverage, at a fraction of the cost of a GeneChip® (currently priced at ˜$450 per chip). However, there is a catch. DNA arrays with microscale features can be read using optical means, such as a fluorescence microscope. Nanoscale features lie beyond the diffraction limit and cannot be optically observed. For a successful whole genome resequencing application, the challenge of reliable readout must be solved. One approach would employ Atomic Force Microscopy (AFM), a method of nanoscale topology imaging. Alternatively, we could attempt to fabricate a DNA Canvas on a gel substrate, that could be uniformly expanded upon imaging, in a method known as Expansion Microscopy [92].

Complex Environments for Molecular Machines

Molecular machines that perform a specific task in response to a stimulus are a key component in all forms of life. Artificial molecular machines are human-engineered nanoscale objects designed to mimic or eventually surpass the roles of existing molecular machines in a controlled manner. The 2016 Nobel Prize in Chemistry was awarded for the design and synthesis of molecular machines that used chemical synthesis to bridge functional chemical groups together. Every task holds information regarding its execution. Traditional electro-mechanical machines, also known as robots, often store an internal representation of the goals, environment, and actions to be executed. When designing robots at a single-molecule level, our current ability to precisely control dynamic and mechanical properties is largely limited. To overcome this difficulty, one strategy is to create simple molecular machines that operate in complex environments [93]. Inspired by the behavior of social insects, such as ants and termites, studies have shown that surprisingly complex tasks can be performed by agents with limited capabilities [94].

DNA is a useful programmable material to build molecular machines. Over the last decade, DNA-based walking motors, also known as DNA walkers, have been designed to traverse multi-step tracks, often embedded on a DNA Origami grid [93, 95]. Furthermore, DNA walkers have been constructed to perform tasks such as cargo-sorting [96] or maze-solving [97]. DNA robots are not the only means where DNA is used to solve complex tasks. Molecular computing is an active research field where pure DNA is designed to form digital logic gates [98] and even neural networks [99]. Recently, a spatial architecture where DNA circuit elements are co-localized on a DNA origami flat surface had shown promise as a new approach for fast and modular molecular circuit design [100].

DNA Canvas holds a few key advantages to DNA Origami as a molecular computing platform. The sheer number of pixels available on a DNA Canvas is beyond the current limits of DNA Origami, allowing more information to be embedded into the environment of the robot/circuit than ever before. Furthermore, DNA Origami is self-assembled in solution and can be difficult to localize. DNA Canvas exists in solid phase on a predetermined location surrounded by visible alignment markers, thus unlocking a range of experiments where an external stimulus or process interacts with the system.

DNA Digital Data Storage

Humanity is generating data at exponential rates, leading to daunting challenges for traditional information storage methods. According to current forecasts [101], global data storage demands by 2025 will exceed beyond the maximal density of any currently available storage method. Traditional digital data storage usually works by changing the properties of materials: electrical properties in flash and phase-change memories or magnetic properties in hard disk drives and tape. Despite the high impact these technologies have made over the last few decades, they are all approaching their density limit. Furthermore, technologies such as magnetic tape pose high maintenance costs. For example, a data center storing an Exabyte (10¹⁸) of data on tape, will require as much as $1 billion and hundreds of millions of kilowatts of electricity to build and maintain for 10 years [102].

Molecular data storage, prominently DNA, has been proposed as an attractive alternative for archival storage, thanks to its extreme density, stable nature, and low energy cost. Storing information into DNA was first demonstrated by artist Joe Davis in 1988, by encoding a 5×7 pixel image to a 28 bases DNA strand [103]. Since then, hundreds of Megabytes [104] have been encoded into DNA, and recent advancements have been extensively reviewed in [101, 105, 106].

DNA Canvas could serve as an attractive platform for DNA information storage.

By rethinking nanopixels as bits, the cost per bit is shifted to depend on sequencing cost, which is both lower and dropping faster compared to DNA synthesis cost. Specifically, a nanopixel fabrication cost is linear in the cost of sequencing ($10⁻⁸ per base [7]) multiplied by a factor (2×oligo length×sequencing depth 100), meaning, a few dollars per “Megabyte” of nanopixels, similar to state-of-the-art. However, following the path to low-cost replication disclosed herein, the cost per nanopixel is disentangled from sequencing, such that the price per chip drops down to the negligible cost of materials and enzymatic replication. Yet, a significant challenge remains. Writing bits to nanopixels by complementary strand hybridization requires nucleic acid synthesis, which brings back the cost to a few dollars per Megabyte. Rather, we could employ nanofabrication tools to write information as spatial patterns. For example, using an electron beam lithography tool that can write 10 nm features, we encode data to a pattern by disabling a plurality of nanopixels that form the encoded pattern. Then, the reference DNA Canvas can be amplified and stored along with a copy that contains the erased nanopixels. Sequencing the two copies enables information retrieval, at the cost of sequencing. Overall, this method implies the cost per written bit is the cost per nanopixel multiplied by the number of nanopixels per feature. For 10 nm features, that factor is just ˜16, yet for 100 nm features it is ˜1600. Furthermore, the cost of the machine, as well as the writing speed (Mb/sec), has to be taken into account, as tools that dynamically write 10 nm features are currently in the price range of >$100K.

Proof of Concept

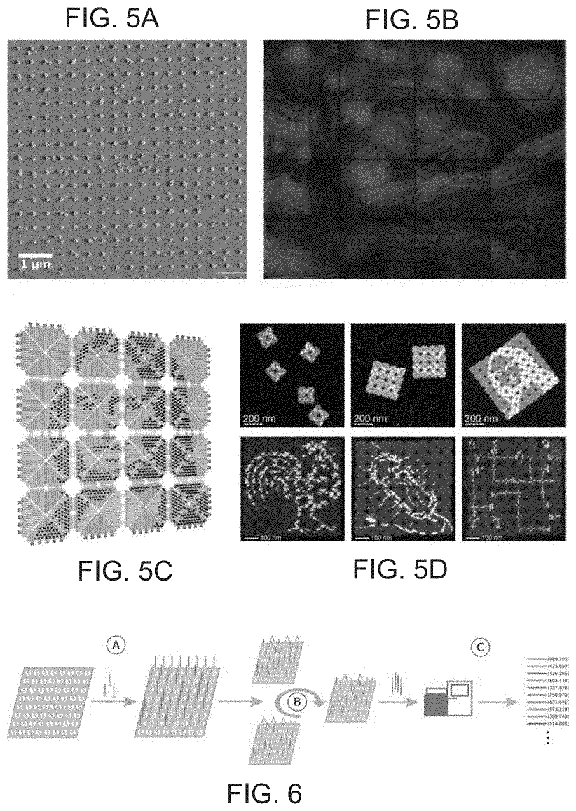

Inspired by Gopinath et al. 65,536 pixels Starry Night [24] and Tikhomirov et al. 8,704 pixels Mona Lisa [26] (FIG. 5C), the first demonstration for DNA Canvas may be a 1M pixels painting. Similar to DNA origami demonstrations, Atomic Force Microscopy will be employed to visualize the end result. Another proof-of-concept that can be optically seen is by patterning a DNA Canvas to create holographic elements. Nanoscale changes in specific patterns cause diffraction of visible light and thus can be seen at a micro- or macro-scale. Beyond these proofs-of-concept, the path diverges. Each potential application, from digital information storage to genomic analysis for medical needs requires an implementation layer over a generic DNA nanoarray.

Barcode Length Versus Number of Binding Sites

A barcode is a string composed strictly of the letters A/C/G/T. A barcode of length k has 4k unique possibilities. We define m has the number of available binding sites. Assuming that each binding site is randomly assigned a barcode i.i.d. The probability p_{m,k} of having a non-unique barcode is:

$${p}_{m , k} = \frac{( \begin{matrix}{4}^{k} \\ m\end{matrix} ) m !}{{4}^{k m}} = \frac{{4}^{k} !}{( {4}^{k} - m ) ! {4}^{k m}}$$

Proof by counting.

Now we prove that for any choice of m>1 and a probability P, there exists

k<<m such that p_{m,k}≤P

Proof:

Let A>0. Choose k=A logo m 4k=mA

$${p}_{m , k} = \frac{{m}^{A} !}{( {m}^{A} - m ) ! {( {m}^{A} )}^{m}}$$

We can reorganize the products, and find an upper bound:

$${p}_{m , k} = \frac{{m}^{A}}{{m}^{A}} \cdot \frac{{m}^{A} - 1}{{m}^{A}} \cdot \frac{{m}^{A} - 2}{{m}^{A}} … \frac{{m}^{A} - m + 1}{{m}^{A}} \leq {( \frac{{m}^{A} - 1}{{m}^{A}} )}^{m - 1}$$

$$Define A = - {log}_{m} ( 1 - \sqrt[m - 1]{P} ) .$$

$${m}^{A} = \frac{1}{1 - \sqrt[m - 1]{P}} ⇒ ( \frac{{m}^{A} - 1}{{m}^{A}} ) = \sqrt[m - 1]{P}$$

$$Thus , {p}_{m , k} \leq {( \frac{{m}^{A} - 1}{{m}^{A}} )}^{m - 1} \leq {( \sqrt[m - 1]{P} )}^{m - 1} \leq P$$

$$Hence , for k = - log ( 1 - \sqrt[m - 1]{P} ) : {p}_{m , k} \leq P$$

Biotin-Tagged DNA Immobilization on Streptavidin Coated Surfaces

Mix Biotin-tagged oligonucleotides (2-100 μM) with Micro Spotting Solution (2×, Arraylt Corp.) to get a 1-50 μM 1× oligo-spotting solution. Print 1 μL of the solution to the surface of the Streptavidin coated slide, for a droplet of diameter ˜1.5 mm. Take care that the tip of the pipette does not touch the surface of the slide in any time. This can be achieved by slowly lowering the droplet from the tip of the pipette, until the droplet touches the surface and sticks to it thanks to surface tension. Incubate for 15-30 minutes in room temperature. To prevent droplet dehydration, the incubation is done on top of a wet tissue inside a clean petri dish sealed with film. Next, suspend SuperStreptavidin Microarray Blocking Buffer (1×, Arraylt Corp.) over the entire surface of the slide/chiplet. Incubate for 30-60 minutes in room temperature. Now wash three times in 1× phosphate buffered saline (PBS) by soaking the chiplet/slide in 1×PBS for five minutes for each step and replace the wash buffer between steps. Last, wash for 10 seconds in 0.1×PBS and blow-dry with Nitrogen. Store in 4° C. in a sealed dry chamber.

On-Chip Enzymatic Fluorescent Labeling

Immobilize G-oligos on one chiplet (Positive) and C-oligos on another chiplet (Negative) according to “Biotin-tagged DNA Immobilization on Streptavidin Coated Surfaces protocol, supra. Next, in two PCR tube, prepare on ice the following reaction for a total volume of 50 μl: 1×NEBuffer 2.1, 1 mM dATP, 1 mM dCTP, 1 mM dTTP, 5U DNA Polymerase I Large Klenow Fragment (New England Biolabs), 5 μM dCTP-Cy3 (Jena Biosciences), 10 μM Primer (Synthesized by IDT) and distilled water (dH₂O). Suspend each chiplet in a tube and move to room temperature for 15-60 minutes. Last, wash three times in 1×PBS and 10 seconds in 0.1×PBS followed by nitrogen dry, similarly to “Biotin-tagged DNA Immobilization on Streptavidin Coated Surfaces protocol, supra. Observe the chiplet under a fluorescence microscope with Ex/Em˜550/570.

SEQUENCES
3′ Biotin-tagged

| Type | Sequence (5′ to 3′) |
|---|---|
| Primer | GCCACCTCAGCAATC |
| + | GccatttcactttacctttacccGATTGCTGAGGTGGC- |
| | (HEG)-T₉-Biotin |
| − | CccatttcactttacctttacccGATTGCTGAGGTGGC- |
| | (HEG)-T₉-Biotin |

5′ Biotin-tagged

| Type | Sequence (5′ to 3′) |
|---|---|
| Primer | ggtgtagtgctgaggtcggtggagg |
| + | Biotin-(TEG)-T-(HEG)-TT-(HEG)-Gc-T₂₀- |
| | cctccaccgacctcagcactacacc |
| − | Biotin-(TEG)-T-(HEG)-TT-(HEG)-Cc-T₂₀- |
| | cctccaccgacctcagcactacacc |

Nicking Enzyme Amplification Reaction

Prepare on-ice the following reaction for a total volume of 50 μl: 1× NEBuffer 2.1, 0.5 mM dNTP, 5U Nb.BbvCl, 2.5U Large Klenow Fragment (New England Biolabs), 2 μM Primer, 0.1 μM Template Oligo (synthesized by IDT). Incubate at room temperature.

Quantitative Assay Using Molecular Beacons

To perform a quantitative fluorescent assay, simply add 0.2 μM Molecular Beacon (synthesized by IDT) to the above reaction. For positive control, add 1 μM of the target oligo (synthesized by Genewiz). For negative control, remove either the enzymes or the primer from the reaction.

SEQUENCES

| Description | Sequence |
|---|---|
| Nicking | GGTGTAGTGCTGAGGTCGGTGGAGG |
| Primer | |
| Template | CC-T₂₀-CCTCCACCGACCTCAGCACTACACC- |
| Oligos | (Spacer)-Biotin |
| | Biotin-(Spacer)-CC-T₂₀- |
| | CCTCCACCGACCTCAGCACTACACC |
| Molecular | (6-FAM)-CCGCGC-T₂₀-GCGCGG- |
| Beacon | (Iowa Black® FQ) |
| Beacon | GAAAAAAAAAAAAAAAAAAAAG |
| Target | |

In an embodiment of the present invention, a DNA nanoarray is disclosed, comprising: a first random oligonucleotide sequence of length N, the sequence comprising a linker molecule; a uniform surface comprising a binder that binds the linker molecule, whereby the sequence is randomly immobilized to a monolayer on the surface; and a second through M random oligonucleotide sequence, the second through M sequences also each comprising a linker molecule; wherein N is a large enough to guarantee within a statistical certainty that every sequence on the surface is unique, and whereby each sequence is bound to a different location on the surface; wherein the uniform surface is a microscale spot; and further comprising a physical chip larger than the uniform surface, wherein the physical chip holds the uniform surface and is milliscale; and whereby enzymatic processes may convert local information concerning each sequence and sequence location into a global mapping at high nanoscale precision, to result in a digital file that maps each sequence to a 2D coordinate. The linker may comprise biotin.

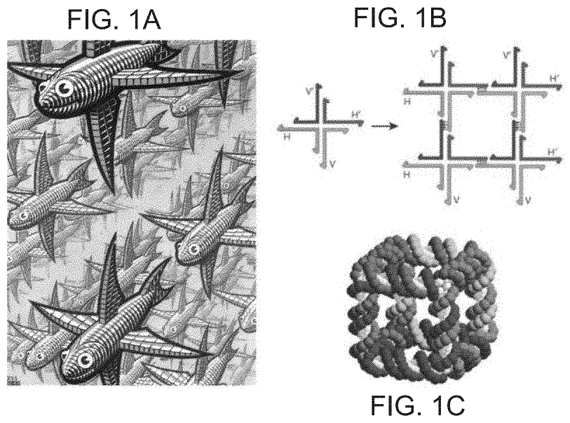

Referring to FIGS. 1A, 1B, 1C, 2-4, 5A, 5B, 5C, 5D, 6, 7, 8A, 8B, 9, 10A, 10B, 10C, 11, 12A, 12B, 13-16, 17A, 17B, 17C, 18, 19A, 19B, 20A, 20B, 20C, 20D, 21, 22A, 22B, 23A, 23B, 24, 25A, 25B, 26A, 26B, 26C, 26D, 27, 28A, 28B, 29, 30A, 30B, 30C, 31, 32, 33A, 33B, 34, 35, 36A, 36B, 37A, 37B, 38, 39, 40A, 40B, 40C, 40D, and 41, FIGS. 1A through 5D are discussed in detail in the Background. FIG. 1A is an image of M. C. Escher's Depth (1955), adapted from [2]. The school of fish crystal-like structure inspired Nadrian Seeman in 1982 to think of DNA as a structural organizer. FIG. 1B illustrates self-assembly of branched DNA molecules into a two-dimensional crystal. FIG. 1C illustrates ligated DNA molecules forming interconnected rings to create a cube-like structure. FIGS. 1B and 1C are adapted from [3].

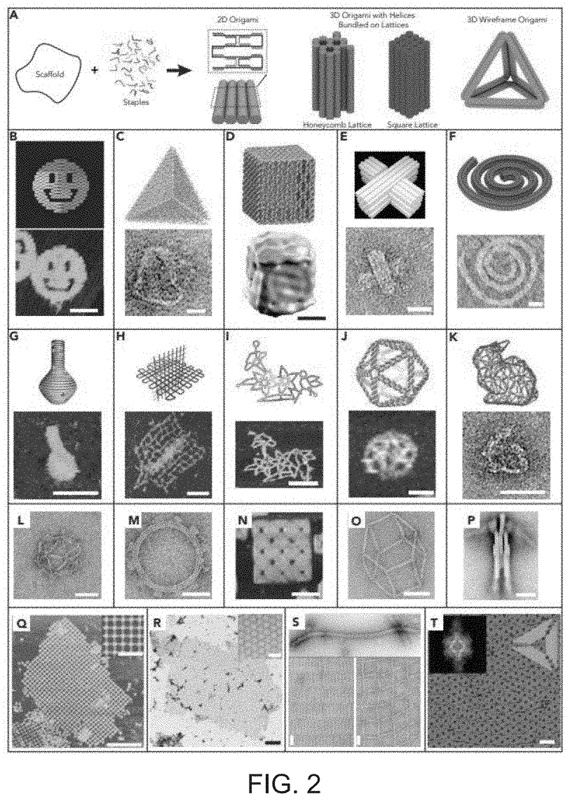

FIG. 2, frames A through T, provide an overview of DNA Origami as reviewed by Wang et al. [4]. Frame A shows DNA origami design strategies for 2D and 3D objects. Frame B depicts a Smiley face. Frame C shows a hollow tetrahedron. Frame D depicts a cube. Frame E shows a slotted cross. Frame F illustrates a curved 6-helix bundle spiral-like object. Frame G shows a nanoflask. Frame H depicts a 3D gridiron structure based on DNA four-arm junctions. Frame I is a wireframe flower-and-bird pattern. Frame J shows a wireframe icosahedron. Frame K depicts a wireframe rabbit. Frame L shows an icosahedron assembled from three origami units. Frame M illustrates a 12-tooth gear assembled from four origami units. Frame N presents a cookie-like superstructure with nine square origami nanostructures assembled along a preformed frame. Frame 0 shows a hexagonal prism assembled from 12 DNA origami tripods. Frame P depicts a robot assembled from three origami precursors. Frame Q is an image of a 2D lattice. Frame R shows a honeycomb 2D lattice. Frame S illustrates dynamic 1D and 2D lattices. Frame T depicts a 2D lattice of a triangle motif. Scalebars are as follows: 20 nm (Frames C-F, K, and P), 50 nm (Frames B, H, J, M, and S), 75 nm (Frame G), 100 nm (Frames I, L, N, O, and R inset), 1 mm (Frame Q), 300 nm (Frame Q, inset), 500 nm (Frame R), and 200 nm (Frame T).

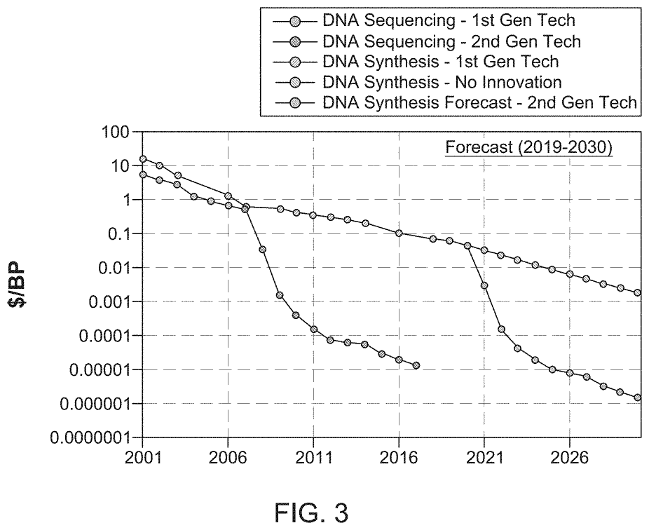

FIG. 3 is a graph of projected costs (in 2018) of DNA sequencing and synthesis, by Potomac Institute of Policy Studies [11]. Lines through alternating solid and broken line (rising to the right) patterned and grid patterned data points represent DNA Sequencing cost trends before and after technological innovation (NGS), respectively. DNA Synthesis: The line having data points with a diagonal line pattern rising to the right represents DNA synthesis cost per base historical trend. The line with data points having a diagonal line falling to the right projects this trend without significant innovation. The line having data points with an alternating solid and broken line pattern falling to the right is a projection that assumes an innovation (similar to NGS) for DNA synthesis, and the resulting cost per base. Adapted from [11].

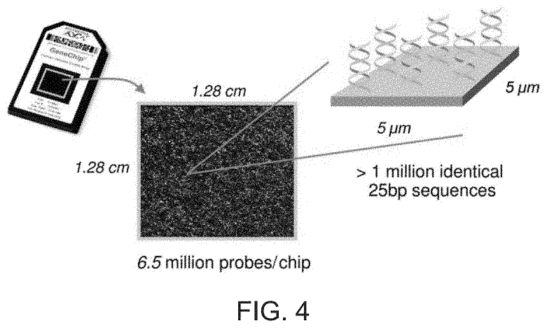

FIG. 4 is a schematic diagram of an Affymetrix GeneChip®, a commercial DNA Microarray platform contains millions of features (25-mer) in a thumbnail-sized chip. Adapted from [14].

FIGS. 5A through 5D show state of the art of Uniquely Addressable DNA Nanoarrays adapted from [23, 24]. Direct Origami Placement (DOP) is illustrated in FIGS. 5A and 5B. FIG. 5A depicts an Electron-beam lithography fabricated microscale grid composed of DNA origami triangles. Each triangle contains >100 uniquely addressed staples of ˜6 nm resolution. FIG. 5B is an image of a 65,536 DNA pixel rendering of Van Gogh's Starry Night. Each pixel is a ˜250 nm cavity fabricated by top-down lithography. Each cavity contains a predetermined quantity of DNA nanostructures equipped with a fluorescent dye, thus digitally varying the emission intensity seen at a microscopic scale. Fractal Assembly is illustrated in FIGS. 5C and 5D, adapted from [26]: FIG. 5C shows 4×4 DNA origami tiles that form a ˜2K pixels Mona Lisa. FIG. 5D is a series of AFM images of fractal-assembly DNA patterns. From top left to bottom right, the images show 2×2 tiles Mona Lisa (˜93% yield); 4×4 tiles Mona Lisa (˜48% yield); 8×8 tiles Mona Lisa (˜4% yield); a rooster; a bacterium; and a photoreceptor circuit.

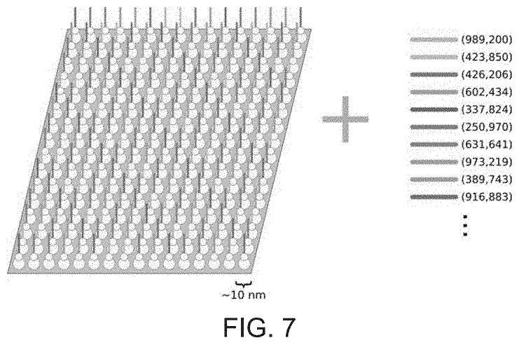

FIG. 6 illustrates a DNA Canvas General Fabrication Scheme. A vast library of oligonucleotides (oligos) may be synthesized using mixed bases at a fixed cost. The random sequence part is denoted as a “barcode”. Each oligo is synthesized with (attached to) a linker molecule (e.g., Biotin), and the solution is suspended over a uniform surface to bind the linker (e.g., Streptavidin-coated glass). Then, by self-assembly, oligos are randomly immobilized to a uniform monolayer on the surface (see FIG. 6, step A). If the barcode length is long enough, it is statistically guaranteed that every barcode on the surface is unique. Essentially, we have fabricated a large-scale DNA nanoarray, wherein each “nanopixel” on the surface is an oligo with a unique address (barcode) and location. Alas, due to the stochasticity of the self-assembly process, we have no information about the addresses and/or locations. Fortunately, recent developments in the emerging field of DNA Microscopy [27, 28] allow us to reconstruct the missing information up to a certain resolution. Briefly, by iterating through a set of co-localized enzymatic reactions (FIG. 6 step B), local proximity information is written and amplified for each oligo in the form of DNA copies we call ligation events. Next, a sample of these ligation events is sent to next-generation sequencing (FIG. 6 step C). If we sequence an ample amount of ligation events, the location and address for each oligo can be reconstructed at nanoscale resolution. Thanks to the dramatic decrease in sequencing costs, this step is financially feasible. Last, the sequencing result is converted using a computational algorithm that converts local information to a global mapping at high precision, such that each barcode is mapped to a nanometric precise location. The result, shown in FIG. 7, is a physical chip coated with uniquely barcoded oligonucleotides+a digital file that maps each barcode to a nanometric spatial location.

Simulation Model

The following base assumptions were made. The core idea behind a DNA Canvas is iterative amplification of adjacent barcoded oligonucleotides, such that each barcode can be precisely mapped to a spatial location. The oligos are immobilized to the surface. For the purpose of in-silico simulations, we define the 2.5 nm Streptavidin-Biotin (S-B) model, shown in FIGS. 8A and 8B, as the means of immobilization. Streptavidin is a tetramer protein that exhibits extremely high affinity to ligand protein Biotin [31]. The diameter of a Streptavidin tetramer is approximately 5 nm [32]. A tetramer has four binding sites and can simultaneously bind up to four Biotin molecules. However, the Streptavidin-coated slides, e.g., supplied by MicroSurfaces Inc. are manufactured such that each Streptavidin tetramer has two sites that are bound to the glass surface, as shown in FIG. 8A, wherein triangles represent Biotin and curves represent oligonucleotides. Hence, each molecule of diameter 5 nm has two available binding sites. We assume the Streptavidin molecules are tightly packed across the surface with no gaps. It should be noted that the molecular weight of either Biotin or oligonucleotides is significantly smaller than Streptavidin by a few orders of magnitude (Streptavidin: 53 kDa, Biotin: 244 Da, Oligonucleotides: #nucleotides×303.7 Da), such that the size of Streptavidin is the major factor determining DNA Canvas resolution density. For the sake of visual clarity, Streptavidin is portrayed herein as a similar-sized molecule to Biotin and nucleotides, with Streptavidin molecules having a gap of 2.5 nm between each other, as shown in FIG. 8B, which illustrates a simplified (disproportionate) visual model. Streptavidin (the binding site) is represented by an S circle. Biotin is a B circle, and nucleotides are circles with their appropriate base letter (A/C/G/T). The barcode region is bolded. The distance between oligos is 2.5 nm, which corresponds to the approximate distance between two available binding sites in a 5 nm Streptavidin tetramer.

Furthermore, we assume two populations of oligos, named 5′ and 3′. These correspond to the location of the Biotin tag in relation to the DNA strand. When performing a ligation step, we assume 5′ strands can strictly bind to 3′ strands and vice versa. Moreover, we assume enzymatic reactions between neighboring pairs have no bias—every oligo is free to ligate to any adjacent strand within reach, without bias.

Moreover, we expect a uniform distribution of oligos on the surface. Meaning, at any given area of a specific size we expect to see a similar number of oligos. There should be no clusters and no gaps. This assumption holds for both populations of oligos: there should not be 5′ clusters or 3′ clusters.

Evaluation

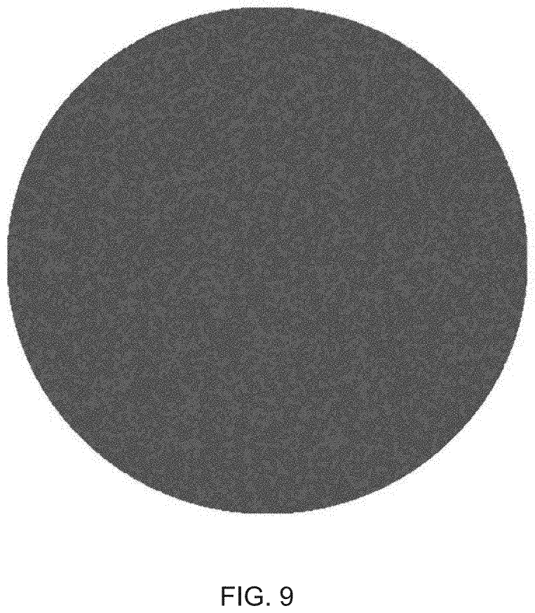

With the aim of evaluating various algorithms for this setting, we have constructed a computational evaluation framework. First, we build a grid. The grid is composed of 2.5 nm cells according to the 2.5 nm S-B model. Next, oligos are randomly generated by classic dart-throwing. Graph drawing algorithms are prone to inaccuracies around corners. Therefore, we choose the shape of DNA Canvas to be a circle (the same circular shape would later be fabricated). Each oligo is randomly assigned to be either 3′ or 5′. See FIG. 9, illustrating a simulation of a circular DNA Canvas of diameter 1 μm containing 119,380 oligos, assuming the 2.5 nm S-B model. Red dots represent 5′ oligos. Blue dots represent 3′ oligos. Last, edges are sampled by a random process, using for example a dart-throwing algorithm—the first oligo is randomly chosen, then a 2D direction vector is randomly picked that leads to the second oligo. The direction vector magnitude must be smaller than the maximal reach distance. The edge is stored if the two oligos are from separate populations (3′ and 5′). The number of edges sampled corresponds to the sequencing depth parameter multiplied by the number of oligos. For example, a simulation with 10K oligos and sequencing depth 2× would contain 20K random edges.

Once the random nodes and edges are generated, the graph is given as input to the evaluated algorithm. Each oligo/node has a known location, the ground truth, that is not revealed to the algorithm. The output of the algorithm is rotated using the Kabsch algorithm [43]. Next, the root-mean-squared-deviation (RMSD) between the ground truth and the aligned prediction is calculated. Numerous random graphs are generated to calculate a confidence interval. As a means to evaluate the accuracy of different methods under a controlled setting, the following parameters are fixed such that local adjacency information is not scarce: sequencing depth: 10×, reach distance: 21 nm (=50 bases long oligos), compactness: 95%.

Graph Connectivity

Connectivity is an important property in graph theory. For DNA Canvas, when the graph is disconnected, the subsequent realization problem becomes fundamentally harder, since there is limited information on how to place the components with respect to each other. According to random graph theory, connectivity can be estimated by the average node degree (d), as first demonstrated in the seminal work by Erdös and Rényi [35]. In our scenario, we can approximate (d) in two ways. On one hand, we define N_{LR} as the number of ligation rounds. In each round, a single edge might be generated per node (up to a constant factor determined by the fidelity of the enzymes involved). Therefore, (d)≤N_{LR}. On the other hand, the parameter sequencing depth, denoted N_{SD}, defines how many reads are sequenced as part of the next-generation sequencing process. Specifically, the total number of reads is N_{SD}·|V| where |V| is the number of nodes in the graph. Thus, the average node degree is

$$\frac{{N}_{SD} \cdot \vert V \vert}{\vert V \vert} ⇒ 〈 d 〉 = {N}_{SD} .$$

By combining these two equations we get [symbol] d [symbol] =min (N_{LR}, N_{SD}).

Practically, performing more ligation rounds endures no extra cost, except for the time of the lab technician (approx. one hour per round). Conversely, raising the sequencing depth leads to a linear rise in sequencing cost, which is the major portion of the total fabrication cost. Therefore, we can safely assume N_{LR}>N_{SD}⇒N_{SD}=min (N_{LR}, N_{SD})=(d). Henceforth, we will explore sequencing depth as the major “knob” influencing graph connectivity (and later, reconstruction accuracy), by strictly setting the number of ligation rounds higher than the sequencing depth.

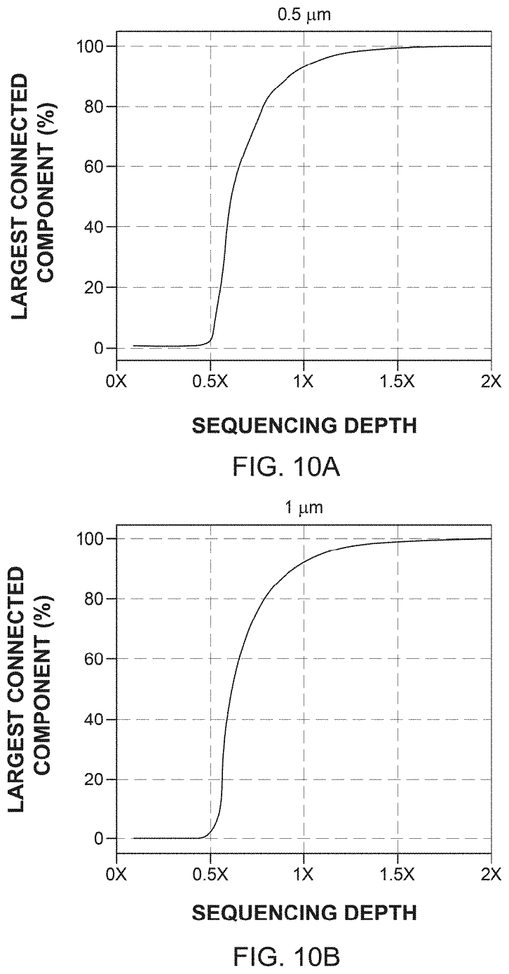

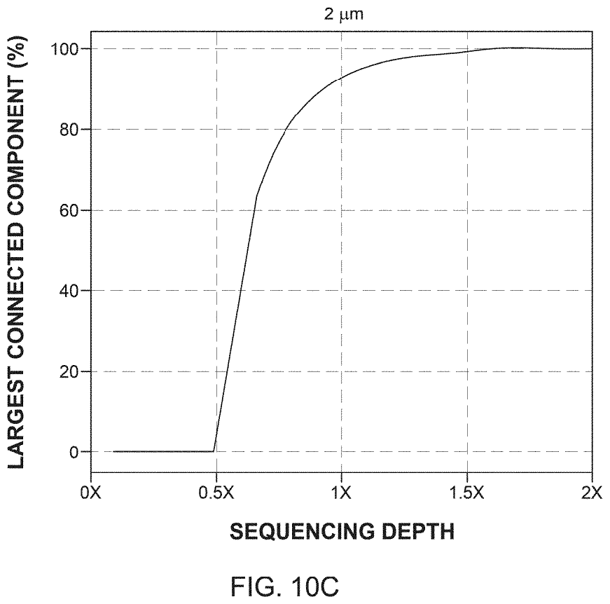

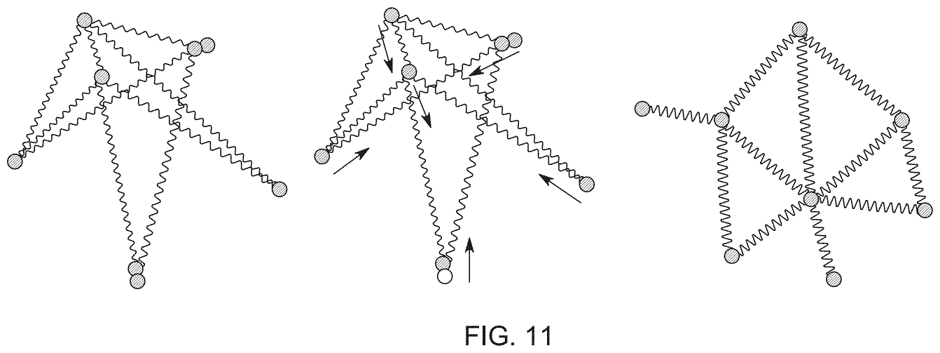

FIGS. 10A, 10B, and 10C are graphs of a largest connected component as a function of sequencing depth, demonstrating the effect of sequencing depth on graph connectivity for various sizes. The Y axis corresponds to the percentage of nodes that belong to the largest connected component in the simulated graph. The graphs are generated as a circular DNA Canvas with diameter d according to the 2.5 nm S-B model (see FIG. 9). FIG. 10A: d=0.5 μm (≈30K binding sites); FIG. 10B: d=1 μm (≈120K binding sites); FIG. 10C: d=2 μm (≈480K binding sites). As a result, sequencing with a depth of 2× (meaning, the number of reads is twice the number of binding sites) would statistically guarantee that our constructed graph would have a single large, connected component, regardless of the DNA Canvas size.

Graph Drawing

Graph drawing is a class of algorithms, where the input is a graph and the output is a 2D embedding that is visually aesthetic. While there is no formal definition for graph aesthetics, it is generally agreed that an “aesthetic” graph is one where there is minimal edge crossing and vertices are uniformly distributed.

A prevalent class of graph drawing algorithms is force-directed graph drawing, where the graph is modeled as a physical system of bodies and forces that act between them, usually as a spring model. The algorithm proceeds to find a placement of the bodies by minimizing the energy of the system. FIG. 11 illustrates a spring model-based graph drawing. Starting from a random position, the graph is treated as a spring system looking for a stable configuration. Adapted from [36].

Fruchterman and Reingold [37] method models the graph as a system of springs, such that a spring applies an attractive force between every two neighboring nodes, and at the same time, a repulsive (“electrical”) force exists between all nodes. Kamada and Kawai [38] method assumes springs between every pair of nodes in the graph, where the length of a spring is associated with its graph distance. In force-directed methods, vertices are slowly moved by the forces acting on them, usually with a decreasing step size. Unfortunately, these methods suffer from high-computational complexity for large graphs. Where |V| is the number of nodes and |E| is the number of edges in the graph, Fruchterman and Reingold requires O(|V|2) calculations per iteration, while Kamada and Kawai complexity is O(|V∥E|). Walshaw [39] proposed a multilevel algorithm, where vertices are hierarchically grouped to form clusters, which define a coarser graph. Each graph is drawn, starting at the coarsest and ending at the original. While Walshaw's method is able to run on large (>250K nodes) graphs in a matter of minutes, it ignores long-range forces between the original vertices. Hu [40] offers an efficient, high-quality improvement to Walshaw's—Scalable Force Directed Placement (SFDP). Briefly, SFDP applies a similar multilevel approach to overcome local minima, while utilizing a Barnes and Hut [41] octree data structure to efficiently approximate short and long-range forces. Moreover, SFDP includes an adaptive cooling scheme to further improve the quality of results over alternative force-directed methods.

Another approach to graph drawing is by high dimensional embedding [42]. First, m pivot nodes are chosen. Next, the shortest travel distance from the m pivot nodes to all nodes on the graph is calculated. Thus, every node now has an m-dimensional embedding. Then, principal component analysis (PCA) is applied to reduce the dimensionality (in our case—to a 2D embedding) while preserving as much variation as possible. High dimensional embedding approach offers very fast running times and straightforward parallelized implementation.

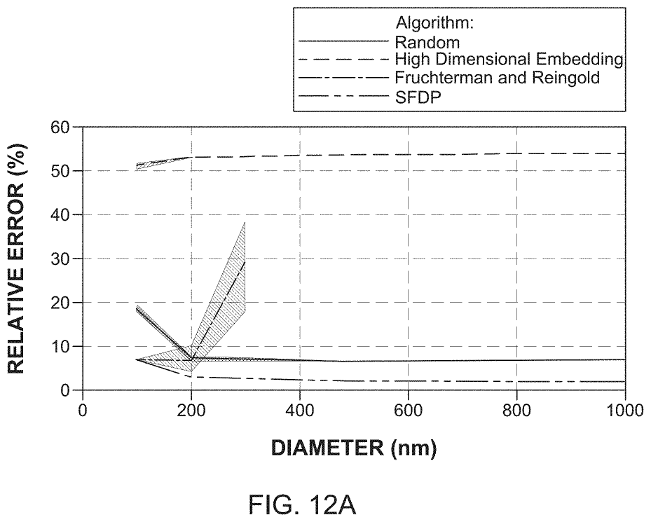

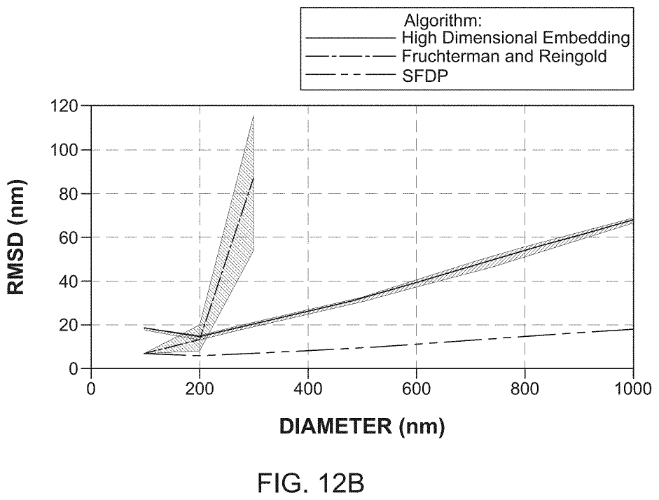

FIGS. 12A and 12B demonstrate the evaluation results of graph drawing algorithms. Each algorithm is evaluated on simulated graphs of various diameters. Shaded areas represent 95% confidence intervals. FIG. 12A shows the relative error (RMSD/diameter). FIG. 12B shows only the evaluated methods with RMSD as the accuracy error. Random method acts as a baseline, where each node is assigned a random coordinate from a uniform distribution. As expected, the relative error is roughly 50%. Fruchterman and Reingold [37] does not scale well (in complexity) for large graphs. High dimensional embedding [42] offers extremely fast running times, even on large graphs, yet the RMSD error is relatively high, significantly larger than SFDP. As a reminder, we compute a mapping precisely once per DNA Canvas, so real-time computation speed is not a requirement in our setting. Therefore, SFDP [40] is a preferred method out of the methods compared, with reasonable running times and consistently low RMSD error (<2.5%).

Henceforth, we apply SFDP to reconstruct global locations from pairwise measurements.

Sequencing Depth

The term sequencing depth is taken from Next-Generation Sequencing (NGS) technology. It is defined as the average number of times a particular nucleotide is represented in a collection of random raw sequences [44]. Sequencing depth acts as a mean to control the inherent errors in the sequencing process. Consequently, the deeper the sequencing, the better the accuracy and completeness of the genomic analysis. Deeper sequencing directly implies higher sequencing costs. A common choice of sequencing depth for NGS applications is 20×.

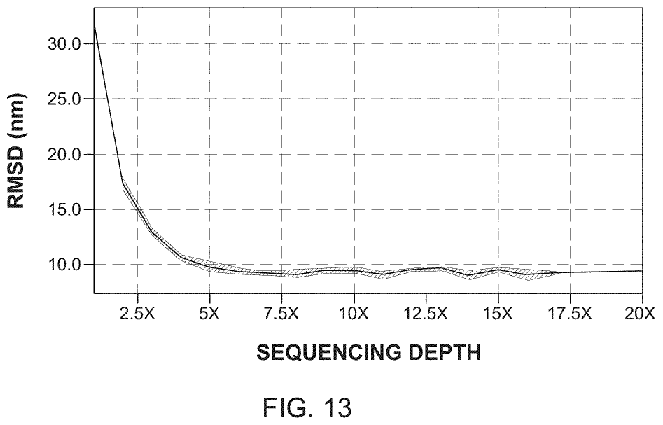

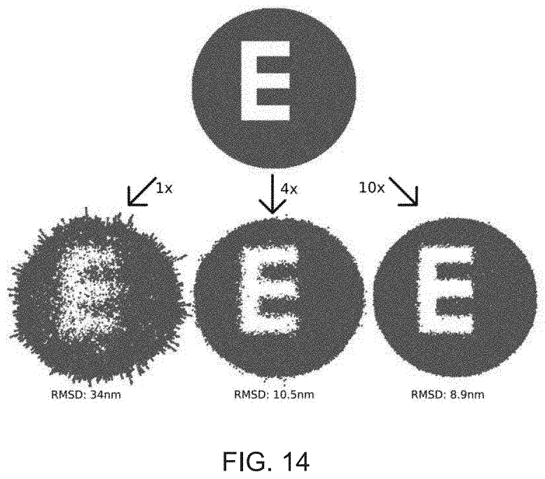

For DNA Canvas, we borrow the term and define it as the average number of times a particular oligo is represented in the collection of random raw ligation events. As seen in FIGS. 10A, 10B, and 10C, a sequencing depth of at least 1.5× is required to ensure the resulting graph is connected. FIG. 13 explores further how sequencing depth affects the mapping localization accuracy. The shaded area represents a 95% confidence interval. For a simulated DNA Canvas of diameter d=0.5 μm (30K nodes), the RMSD drops significantly as the sequencing depth increases from 1×, yet from a sequencing depth of 7×, the RMSD error plateaus around 9 nm. Intuitively, this result affirms that more edges improve the method's localization only up to a certain point, where the abundance of edges per node does not add new information. FIG. 14 is a visualization illustrating the relationship between sequencing depth and RMSD (mapping accuracy). On top, a simulated DNA Canvas graph (d=0.5 μm, 30K nodes) has blue dots representing 5′ oligos and red dots representing 3′ oligos. A pattern (the letter ‘E’) is drawn in white for illustration purposes. Below, SFDP method output given the top graph structure as input. The three inputs differ only by sequencing depth, and consequently, mapping accuracy (measured in RMSD). From left to right: 1× (30K edges), 4× (120K edges), 10× (300K edges). Notably, sequencing depth is directly related to sequencing costs and therefore the total DNA Canvas cost, as sequencing is the costly part of the process. Hence, an optimal trade-off between cost and resolution may be selected with regard to the DNA Canvas application's requirements. Thus, the curve in FIG. 13, from a sequencing depth of 1× up until sequencing depth 8×, essentially acts as a Pareto frontier [45]—the set of optimal trade-offs between cost and resolution.

Compactness

One of our key assumptions is that Streptavidin molecules are tightly packed across the surface. Yet, we have so far assumed that Biotin-tagged oligos are occupying all available binding sites (100% compactness). In practice, we can adjust the compactness of oligos by introducing a competitive ligand (e.g., Biotin without oligo). For the purposes of simulation, the compactness is adjusted between 1% to 99%.

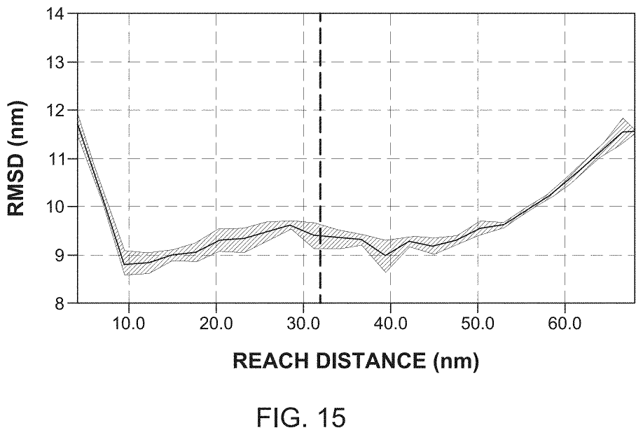

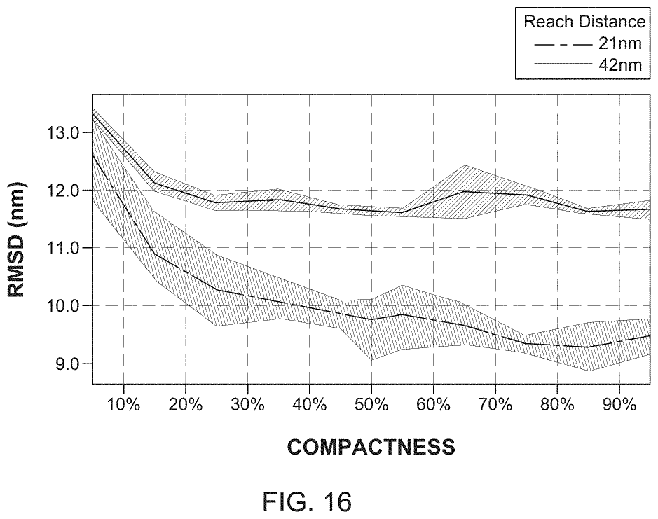

As expected, when the compactness is low (<25%), the accuracy error increases, since local adjacency information is sparse. Notably, after a certain point (>75% for reach distance of 21 nm, 25% for 42 nm), the compactness has no significant effect on the RMSD. Similar to sequencing depth, this insight has important implications both for hybridization efficiency and fabrication costs. Sufficient spacing between oligos has been known to increase hybridization and enzymatic efficiency, which are both important steps in fabricating as well as using a DNA Canvas device. Moreover, the denser the array of oligos, the more edges we need to sequence, thus entailing higher sequencing costs. Therefore, DNA Canvas applications can choose a compactness setting as a second trade-off between price and precision, along with sequencing depth. Additionally, FIGS. 15 and 16 elucidate an important design concept for DNA Canvas—for high-precision applications, the oligo length (and the derived reach distance) should be minimized.

FIG. 15 demonstrates the effect of varying reach distance on the localization accuracy for a simulated DNA Canvas: d=0.5 μm, 30K nodes. The shaded area represents a 95% confidence interval. The dashed line represents the practical minimal reach distance for oligos to undergo iterative proximity ligation. Interestingly, the relationship is not linear. If the reach distance is too short, each node in the graph would have very few neighbors, leading to poor precision. If the reach distance is too far, each node will have many neighbors. Since each node is localized in relation to its neighborhood with respect to the rest of the graph, having too many direct neighbors results in a fuzzy outcome.

FIG. 16 illustrates the effect of compactness on mapping accuracy for a simulated DNA Canvas: d=0.5 μm, 30K nodes. Shaded areas represent a 95% confidence interval. The broken line corresponds to oligo length of 50 bases (reach distance: 21 nm). The solid line corresponds to 100 bases (reach distance: 42 nm).

Also envisioned is a computational workflow that explicitly abides by the uniformity of DNA Canvas, along with other assumptions, for example by binning.

Reach Distance

Each edge in the graph is originated from a ligation and amplification event of two oligos immobilized to the surface. Therefore, two oligos can ligate only if their ends can reach each other. Reach distance is defined as the maximal distance for which two immobilized oligos can ligate across. Roughly, an oligo composed of k bases is k×0.34 nm long. Thus, the reach distance between two oligos of length k is:

Reach Distance (nm)=2×0.34×k=0.68k

We can adjust the reach distance by adding or removing bases in our sequence design. Alternatively, it is possible to insert special molecules called spacers (e.g., Polyethanolglycol) to increase the oligo's length without adding new bases. As a lower bound, there must be enough bases such that two oligos can reach each other over the gap defined by the Streptavidin molecule structure—at least 6 bases. Furthermore, the oligo sequence must have enough bases to accompany components that are integral to the iterative amplification process: a barcode, priming site, nicking site, restriction site. For barcode length k, the minimal oligo design is approximately 23+k, which corresponds to a reach distance of 15.64+0.68k nanometers. For an upper bound, while it is theoretically possible to synthesize oligos of various lengths, currently vendors that offer oligo synthesis services (IDT, Genewiz), can synthesize oligos up to 100 bases long (for oligos that include complex modifications, such as a 3′ Biotin tag). Therefore, we will examine various reach distances as a function of k in the range of 6-100 bases, which corresponds to reach distances of 4-68 nm.

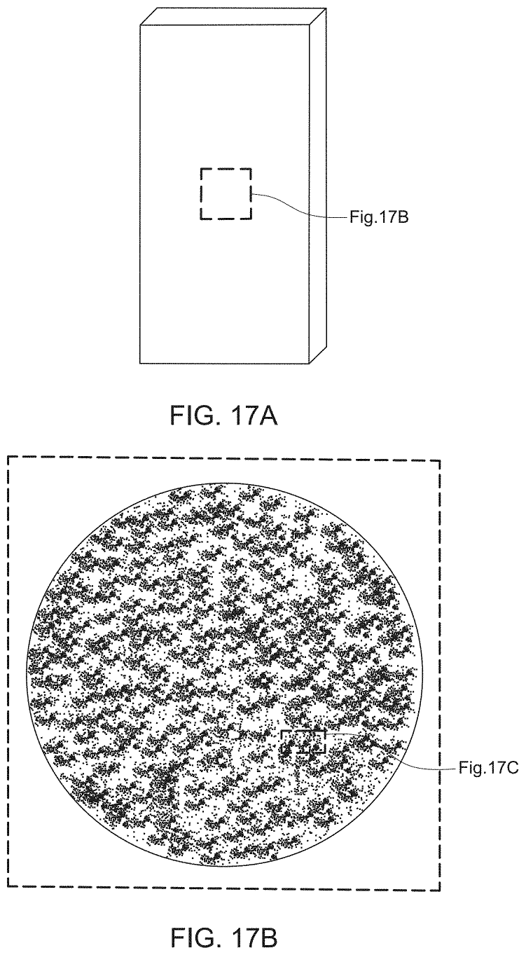

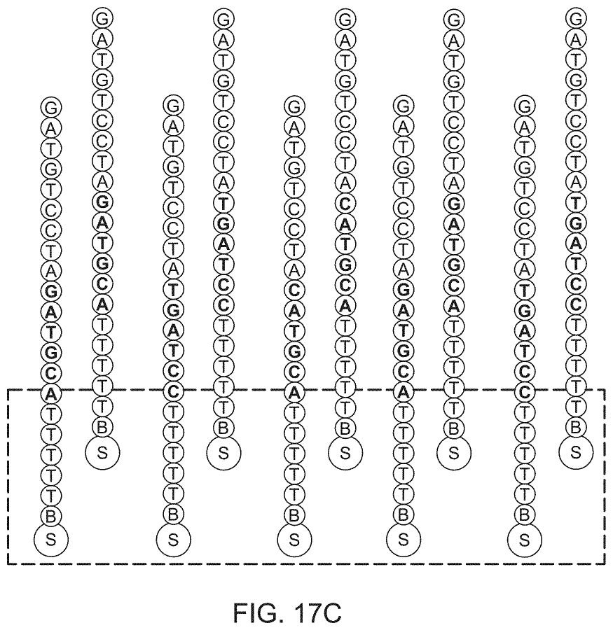

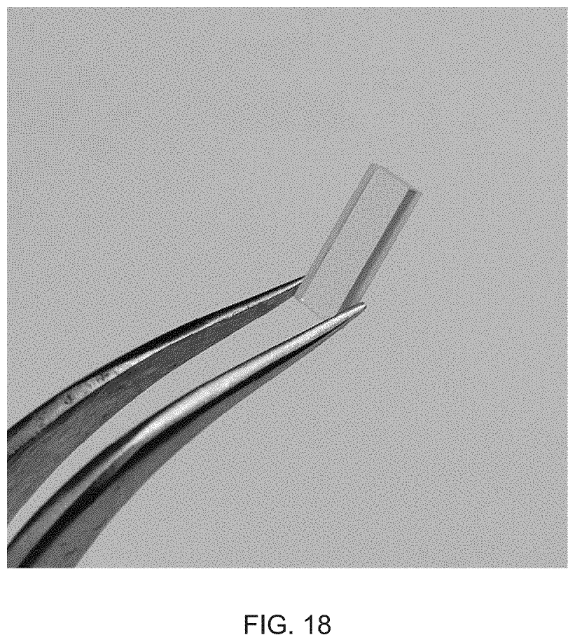

The fabrication process of a DNA chiplet, the milliscale device containing a microscale spot composed of DNA nanopixels, is shown (at multiple scales) in FIGS. 17A, 17B, 17C, and 18. FIG. 17A illustrates a diced glass 3×6 mm slide, designed to fit inside a commercially available PCR tube. The slide has an active area with exactly one 5 μm spot of Streptavidin binding sites and Biotin-tagged oligos, enlarged as FIG. 17B. The stippling in FIG. 17B represents 5′ and 3′ oligos. FIG. 17C illustrates a DNA nanoarray, each strand having a unique barcode, indicated in bold. Alternating strands are 2.5 nm apart. FIG. 18 is a photograph of a 3×6 mm DNA Chiplet.

We present a set of assays to validate the quality of the fabrication process, as well as experimentally adjust some of the “knobs”. Moreover, we present challenges (and solutions) that arise in the intersection between traditional microfabrication and Streptavidin-coated substrates. Our contributions span beyond the scope of a supporting device for DNA Canvas to hold interest to any user interested in nano- or micro-scale patterning of surfaces coated with proteins and/or nucleotides.

Precision Dicing

The alignment markers have an additional benefit. The last step of the fabrication process is dicing the slide into chiplets, such that each spot lies approximately at the center of each chiplet. The alignment markers allow for visual alignment in a dicing saw. The result can be seen in FIG. 18.

It should be noted that the dicing procedure could potentially introduce unwanted particles on the surface of our slide. Therefore, wash and dry steps were done after dicing. The fluorescence based-immobilization assay was then applied to ensure the surface does not exhibit unwanted artifacts.

Fluorescence-Based Immobilization Assay

Uniform density is a key feature of the inventive product. Briefly, if there are islands or holes on the surface, the computational model would fail to localize nanopixels with high precision. Therefore, we use a fluorescence-based assay to test the quality and uniformity of coated slides.

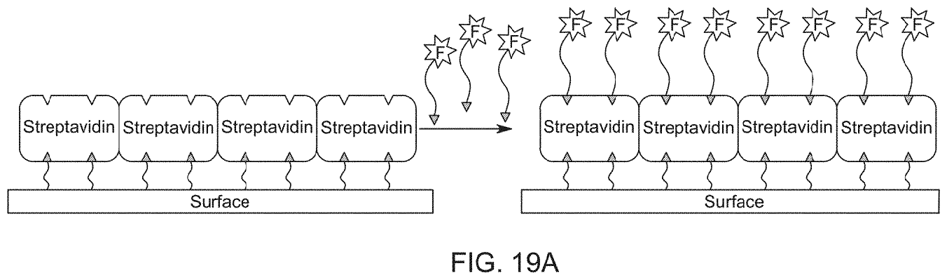

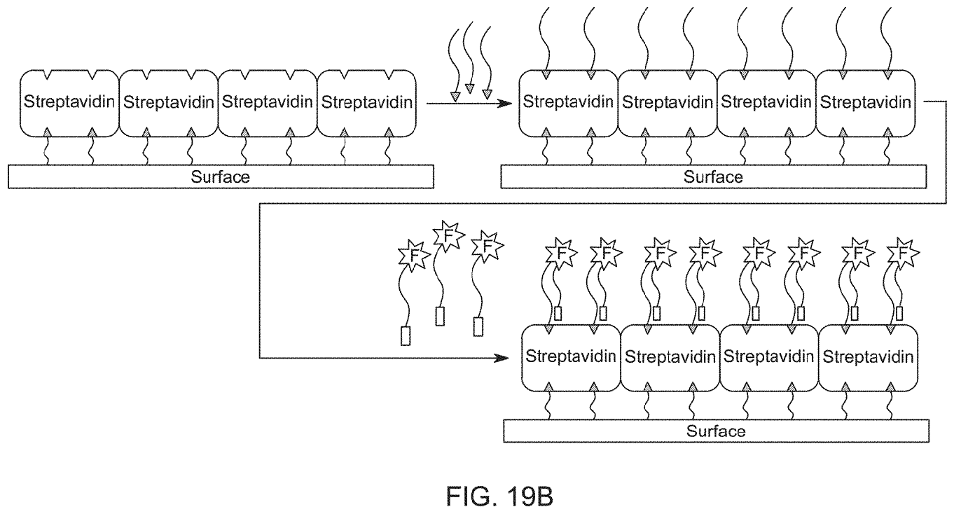

The Fluorescence-based Immobilization Assay (Streptavidin-Biotin Linking) has two versions. In the basic version (FIG. 19A), we suspend a small droplet (1-2 μl) of oligos, shown as curves, that are tagged with a linker (e.g., Biotin, Amine), shown as triangles, and a fluorophore F (e.g., fluorescein [FITC], Cy3), shown in heptagrams. Following the immobilization protocol, the surface is washed multiple times. Next, the surface is observed using a fluorescence microscope (See DNA SEQUENCING AND FLUORESCENCE SAMPLING METHODS).

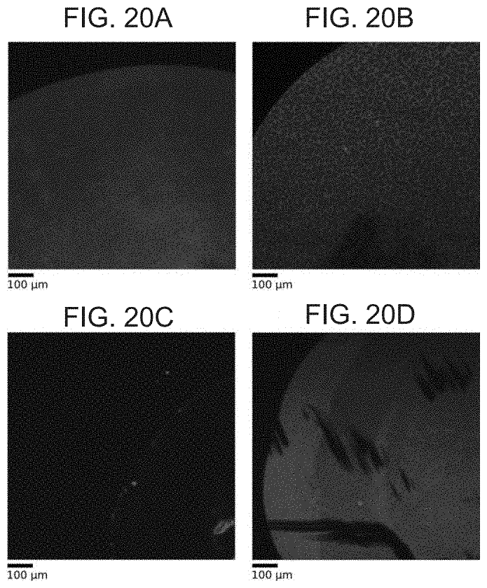

Ideally, a uniform fluorescence signal is present wherever the droplet touched the surface, usually in the form of a circle. The edge of the circle is an attractive feature to observe the difference between the signal to the background noise. FIG. 20A illustrates a good result with strong signal-to-background fluorescence, a crisp round edge, and uniform intensity across the droplet. Negative or “bad” results may appear in the form of a non-uniform surface implied by holes and/or islands as shown in FIG. 20B; a weak fluorescent signal appearing as a “coffee stain” effect, having stronger fluorescence on the edge of the droplet, e.g., due to drying out as seen in FIG. 20C; scratches to the surface shown in FIG. 20D, which are sometimes caused by pipette tip or tweezers; or weak signal-to-background signal. The red color signifies fluorescence intensity, not actual color. The results shown in FIGS. 20A-20D were imaged using a Zeiss™ LSM 5 Pascal confocal microscope at the Center for Bits and Atoms.

Next, we applied the second assay version, a hybridization-based assay (FIG. 19B). Similarly, we suspend a small droplet (1-2 μl) of oligos that contain just a linker and a predetermined sequence (˜30 bases, Tₘ>60 C). The surface is washed multiple times to remove excess oligos. Then, we suspend a larger volume of oligos that contain the exact complementary sequence and a fluorophore. The goal of this assay is to verify DNA hybridization. The readout is the same—a uniform fluorescent circle viewed by a fluorescence microscope.

Using these assays, we have evaluated Streptavidin-coated surfaces from various vendors as well as in-house coated glass slides. Further, we have optimized our immobilization protocol under various conditions (“Biotin-tagged DNA Immobilization on Streptavidin Coated Surfaces protocol, supra). As a result, we choose to use slides from a specific vendor (MicroSurfaces Inc.) as the fluorescence signal and uniformity exhibited on their slides were superior to other vendors as well as our own in-house attempts. The cost per slide is $50 (which is diced into 100 chiplets, thus $0.5 per chiplet), compared to ˜$10 fabrication cost in-house.

A) Oligos tagged with Biotin (green triangle) and a fluorophore (yellow star) are directly immobilized to the surface. B) Two-step hybridization assay. First, Biotin-tagged oligos with a predetermined sequence are immobilized. Next, oligos with the complementary sequence, tagged with a fluorophore are hybridized to the surface.

Microfabrication

In order to attain reasonable fabrication costs, the active area of a DNA Canvas must be less than 10 μm in diameter. A custom microfabrication process using photolithography is disclosed herein. The starting point of the process is a 25×75 mm Streptavidin-coated microscope slide. The output is a set of 3×6 mm chiplets, each with a single microscale spot with active Streptavidin binding sites. Importantly, the rest of the chiplet is nonreactive to DNA immobilization.

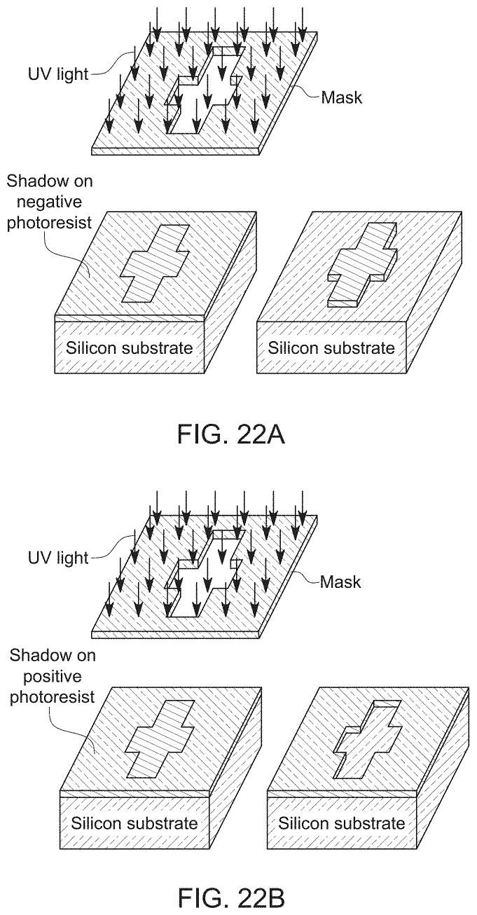

Photolithography is a process where a pattern is transferred to a photosensitive polymer (a photoresist) by exposure to a light source (e.g., ultraviolet [UV]) either through an optical mask (such as shown in FIGS. 22A and 22B, adapted from [63]) or a direct-write technique [62]. In microfabrication by photolithography, the pattern may be further transferred to the substrate by subtractive (etching) or additive (deposition) techniques. Photoresists can be classified as positive or negative. For positive resists, the photochemical reaction that occurs during exposure weakens the polymer, making it more soluble to the developer, so the positive pattern is achieved. See FIG. 22B. For negative resists, exposure to light induces polymerization, such that the negative resist remains on the surface of the substrate where it is exposed, and the developer solution removes only the unexposed areas. See FIG. 22A.

Preliminary Results Using Electron Beam Lithography

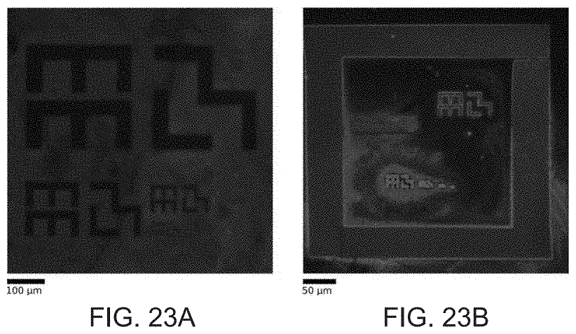

Experiments, in collaboration with Junichi Ogawa at the Massachusetts Institute of Technology (MIT) Media Lab, were performed with a microfabrication process using a negative photoresist (e.g., SU-8) and an Electron Beam Lithography (EBL) tool to pattern Fluorophore-tagged DNA at various micro-scales. The process involved spin-coating a Streptavidin-coated glass slide with SU-8 negative resist and patterning using EBL (in environmental mode) to direct-write microscale patterns. The resist was developed such that the unexposed areas were removed. Then, the fluorescence-based immobilization assay was applied. Biotin-tagged oligos with a fluorophore (Cy3) were suspended and immobilized. Streptavidin binding sites were available only in areas not exposed to the electron beam (as remaining binding sites were still covered by polymerized SU-8). Thus, a microscale crisp fluorescent pattern was achieved as shown in FIG. 23A, which shows good patterning with a nonuniform background signal. Red color signifies fluorescence intensity.

The EBL+SU-8 approach was faced with a few significant challenges. First, the maximum exposure area in the EBL tool was too small to expose the entire chiplet at once. Thus, multiple exposures had to be manually stitched. Furthermore, the thick SU-8 film remaining on the chiplet raised compatibility issues with the downstream biological processes. Specifically, the resist can be “sticky”, especially around the edges of the pattern, such that DNA would unintentionally be immobilized to the resist. See FIG. 23B, which shows various artifacts including bright edges and unwanted fluorophore immobilization, possibly due to “sticky” resist. While the possibility of scaling the DNA Canvas to the nanoscale regime using EBL is attractive, costs and time considerations make EBL-based processes not ideal for fabricating a low-cost microscale solution. FIGS. 23A and 23B were Imaged using a Zeiss™ LSM 5 Pascal confocal microscope at the Center for Bits and Atoms.

New Process Using Photolithography

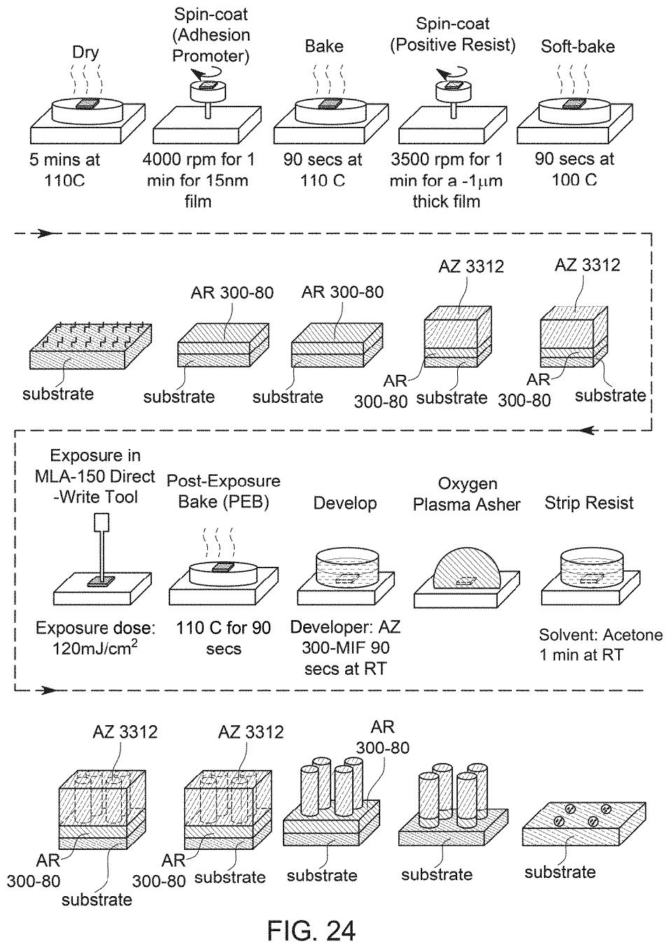

For this reason, we developed a novel microfabrication process for patterning microscale spots on Streptavidin-coated surfaces that is optimized for compatibility for subsequent enzymatic reactions at the Baldo lab at MIT. As shown in FIG. 24 the microfabrication process obtained microscale Streptavidin spots on an otherwise clean surface starting from a uniformly Streptavidin-coated glass substrate and using a positive resist and subsequent plasma ashing.

Importantly, at the end of the process, Streptavidin-coating remains strictly within the active microscale spot, and no resist is left on the surface. Furthermore, we introduce a compatible etching process to generate features that help to find the “invisible” spot under an optical microscope for downstream imaging (e.g., AFM).

To ensure the Streptavidin coating remains reactive to subsequent enzymatic reactions, exposure to ultraviolet (UV) light and/or plasma is minimized and heating steps are limited and shortened as proteins undergo denaturation under elevated temperatures over extended periods of time. Known solvents that are typically used in the art with inorganic substrates proved to be incompatible with Streptavidin coating.

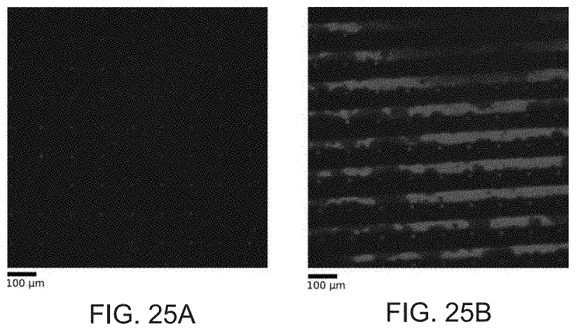

FIGS. 25A and 25B illustrate 10 μm spots arrays post resist stripping and fluorescence labeling according to the inventive microfabrication process. Biotin-tagged DNA linked with a fluorophore (Cy3) is immobilized to the surface. As shown in FIG. 25A, after exposure dose optimization, notice some spots are missing due to lack of adhesion. FIG. 25B illustrates the result of an under-exposed resist. Both figures were imaged using a Zeiss™ LSM 5 Pascal confocal microscope at the Center for Bits and Atoms.

Spots

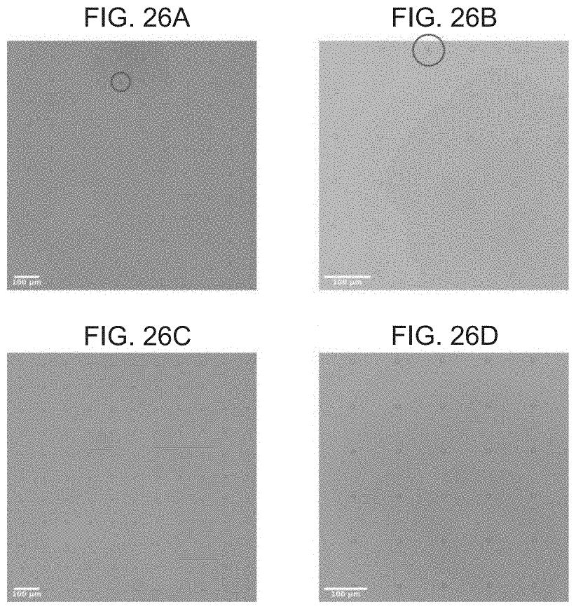

Applicants have discovered that due to the comparatively high surface energy of glass, an adhesion promoter is required for resist application. FIGS. 26A and 26B illustrate a microscale spot array produced without an adhesion promoter, at 5× and 10× magnification, respectively. Note that ˜10% of the spots are missing. A “drifted” spot is marked with a red circle. Given the presence of a Streptavidin monolayer, an adhesion promoter that relies on covalent monolayer binding (such as hexamethyldisilizane [HMDS]) cannot be employed. Therefore, we opted to use a spin-coatable adhesion promoter developed by Allresist (AR 300-80), applied at 4000 rpm to yield a 15 nm thick film. Next, a positive resist (AZ 3312) was spin-coated at 3500 rpm and soft-baked at 100° C. for 90 seconds to form 1 μm thick film. Using the MLA-150 direct-write photolithography tool (Heidelberg Instruments), the desired pattern was transferred to the resist. Here, we exposed all areas around an array of microscale spots. After a post-exposure bake at 110° C. for 90 seconds, the resist was developed in AZ 300-MIF. This left a 1 μm protective “pillar” of resist over each spot. Next, we placed the slide in an Oxygen Plasma Asher for 2-5 minutes at plasma density resulting in etch rates of 12 nm per minute. This step etched all exposed organic matter, meaning both the remaining adhesion promoter and the Streptavidin molecules around the resist pillars were entirely removed. Then, both the adhesion promoter and the resist pillars were stripped using acetone. FIGS. 26C and 26D are optical microscope images imaged in the Nanostructures Laboratory (NSL) at MIT at 5× and 10× magnification, respectively, of 10 μm spot arrays produced using an adhesion promoter (AR 300-80) and developed in a positive resist AZ 3312 according to the method described above. ˜100% of the spots are correctly located.

Typically, according to common wisdom in the art, the preferred stripping agent would be N-Methylpyrrolidone (NMP) or Dimethyl Sulfoxide (DMSO). However, applicants discovered that Streptavidin is incompatible with both by applying the Fluorescence-based immobilization assay and getting no fluorescent signal after using the process with either of those solvents. Therefore, we chose to use Acetone as it exhibited no compatibility issues. Generally, there are concerns with Acetone leaving residue. However, upon imaging, we did not observe any irregularities.

Markers

The spots are invisible under an optical microscope. To observe a DNA Canvas patterning/decoration, Atomic Force Microscopy (AFM) may be used, a technique that allows nanoscale topological imaging. AFM requires spatial alignment under an optical microscope to the region of interest followed by a scanning step that takes several minutes per 1 pmt. Thus, alignment markers visible under an optical microscope are needed to locate the spot. For that purpose, we have developed a second process to generate alignment markers around each spot. Here, we tried a resist-based approach, a lift-off process approach, and a subtractive approach. The resist-based approach left resist on the chiplet, which raised similar issues to the SU-8 approach described above. The lift-off procedure allowed deposition of only 50 nm thick silver film (as opposed to 1 μm resist), introducing a metallic surface in proximity to the spots that gave rise to problems both in fluorescence imaging as well as downstream enzymatic processes. Therefore, we chose the subtractive approach, which introduces no additional materials at the end of the procedure.

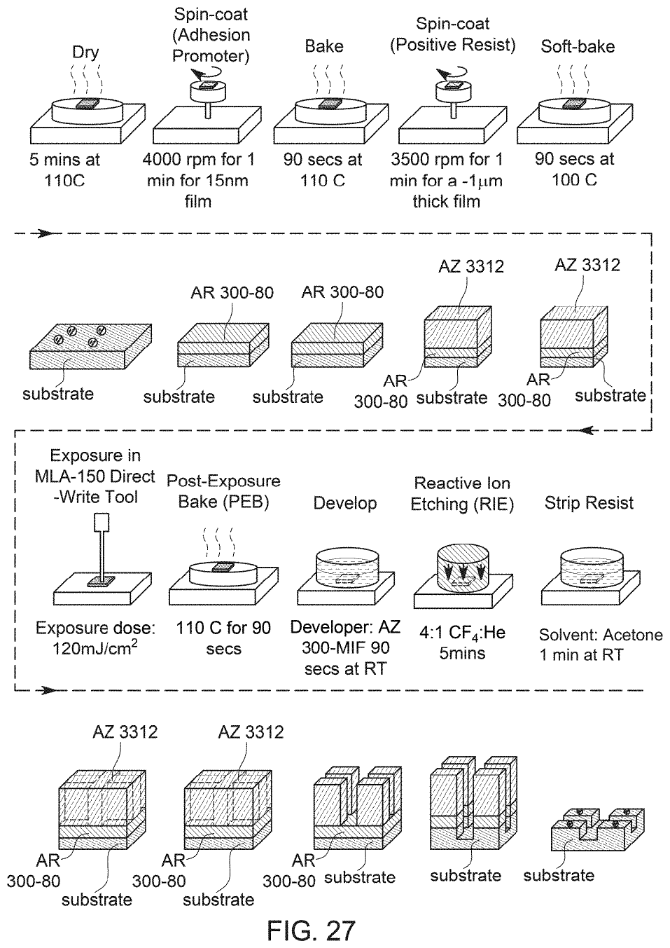

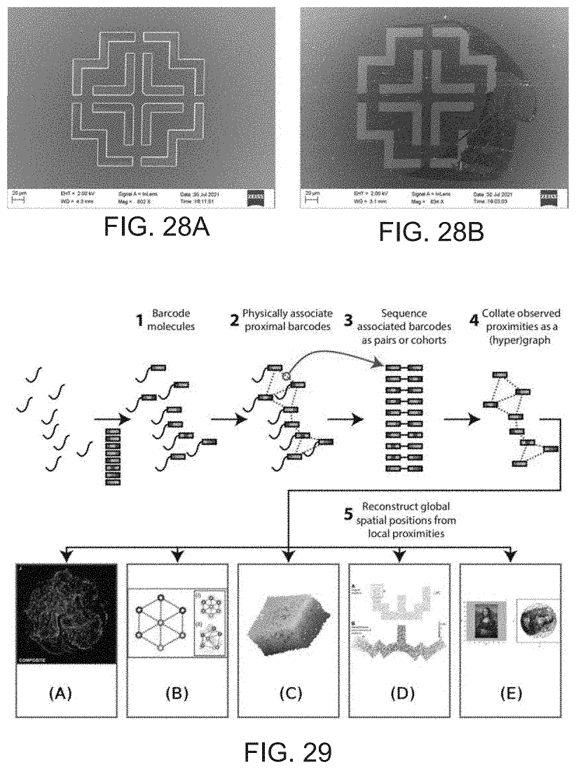

The inventive microfabrication process for patterning optically visible alignment markers, illustrated in FIG. 27, includes many of the same steps as the inventive process described above for spots microfabrication. Starting from Streptavidin spots on an otherwise clean surface and using a positive resist, we expose and develop the area within the markers, leaving trenches behind. Then, placing the developed pattern in a Reactive Ion Etcher (RIE) allows for etching of the desired pattern of optically visible alignment markers around the Streptavidin spots. First, we remove the adhesion promoter with a brief 1:1 O₂:He etch. Then, by using a 5:1 gas mixture of CF₄:O₂, the now exposed glass surface within the trenches is etched. CF₄ is an isotropic reactive etchant, yielding high rates of SiO₂ etch. The addition of small amounts of oxygen optimizes the achievable etch rates [64] to minimize UV exposure of the Streptavidin. FIG. 28A is a scanning electron microscope (SEM) image depicting the markers developed in resist, before etching. FIG. 28B is a scanning electron microscope (SEM) image showing the markers after etching and resist stripping. As can be seen, resist removal is incomplete, thus further measures are preferably taken to improve resist removal in order to obtain a resist-free surface at the end of the process. The images were taken in the Nanostructures Laboratory (NSL) at MIT.

DNA Microscopy

DNA Microscopy [27, 28, 66, 67], is an emerging class of techniques that utilize DNA-based enzymatic reactions to perform optics-free imaging. Prior art microscopy generally relies on photons (e.g., in optical microscopy), electrons (e.g., in electron microscopy), or scanning probes (e.g., in atomic force microscopy) to decipher the spatial arrangement of a given sample. However, these techniques suffer from various disadvantages such as the diffraction-limited resolution in optical imaging, expensive instrumentation in electron beam imaging, and low throughput in atomic force imaging [68]. DNA microscopy relies on stochastic binding of proximal oligos followed by next-generation sequencing as a medium for molecular-scale imaging. DNA microscopy schemes, reviewed in and adopted from [27], are shown in FIG. 29. The techniques follow these general steps: (1) molecules of interest are barcoded with distinct DNA oligos; (2) barcodes from neighboring molecules are physically associated such that (3) they are sequenced as one cohort, with sequencing information directly reflecting their proximity; (4) the individual proximities captured by sequencing are abstracted into a graph, with nodes representing barcoded oligos and edges representing the observed proximities; and (5) computational algorithms reconstruct from this graph the global map of all spatial positions. Examples shown in FIG. 29 include: (A) 10⁵-10⁶ diffused mRNAs in a cell [28]; (B) Dozens of oligos immobilized on a DNA Origami surface [67]; (C) a simulation of Chemical puzzling [29]; (D) a simulation of Oligos on a surface [69]; and (E) a simulation of polonies on a surface [30]

Scheme Overview

In each round, two oligos of opposite polarity are ligated and extension of one of the primers leads to a mobile copy containing two barcodes. Last, a restriction enzyme recognizes the now-full site and cleaves the oligos apart. The ligation and extension step described above repeats, such that in every round, a different pair of oligos may be ligated.

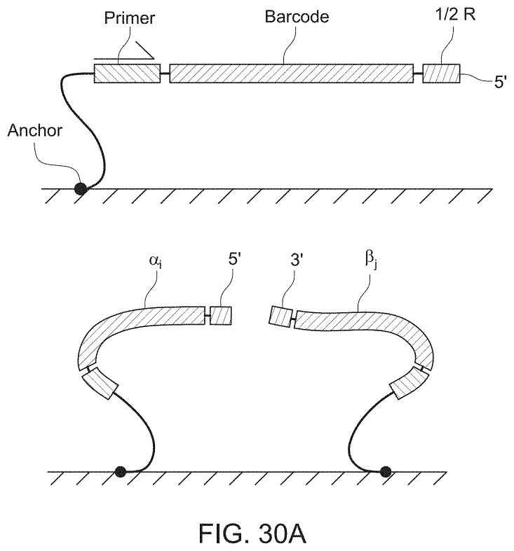

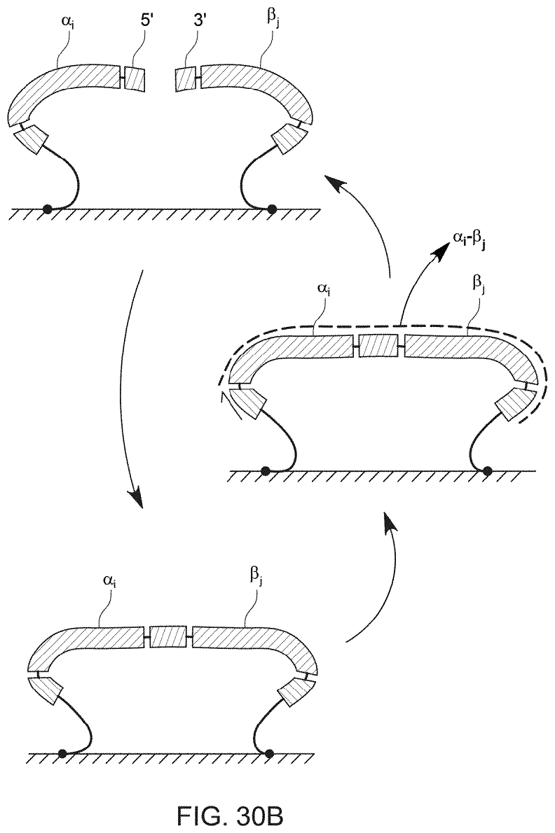

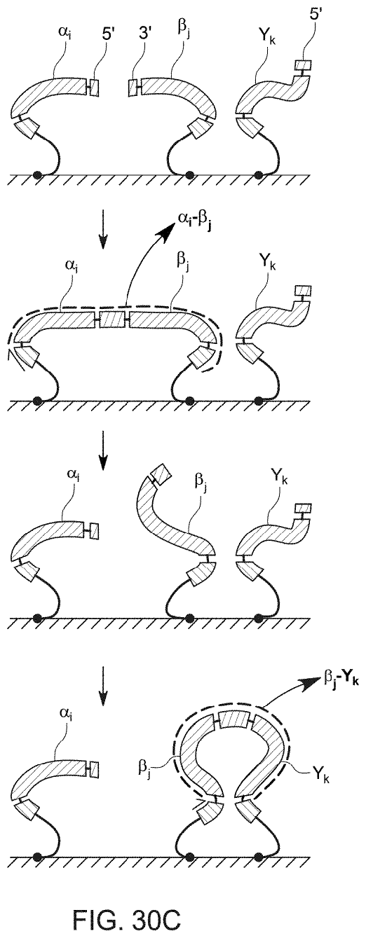

Boulgakov et al. [69] presented a simple approach to DNA microscopy, called Iterative Proximity Ligation (IPL), illustrated in FIGS. 30A, 30B, and 30C. In IPL, two populations of oligos are immobilized to a surface using Streptavidin-Biotin linking chemistry, as shown in FIG. 30A, bottom. Half of the oligos have the Biotin tag on the 5′ end and the other half have the Biotin tag on the 3′ end, such that they are immobilized in an approximately 1:1 mixture. As seen at the top of FIG. 30A, each oligo comprises a functional group for attachment, a primer site, a unique barcode sequence, and a restriction half-site. In each round, illustrated in FIG. 30B, the oligos are ligated by DNA Ligase and a short bridging oligo, followed by extension to duplicate the barcode pair by DNA Polymerase and a primer. At the end of each iteration, a restriction enzyme recognizes the now-full restriction site and cleaves the oligo pair, thus returning to the initial state. This cycle is repeated multiple times, wherein every iteration, a different pair of oligos can ligate as shown in FIG. 30C, by some probability.

We opt for at least eight rounds of ligation and extension to make up for any inefficiencies introduced by the underlying enzymes. One shortcoming of IPL is the number of extensions per cycle. After two oligos are ligated, DNA polymerase and a primer oligo extend the pair to produce a dsDNA copy immobilized to the surface. In order to produce multiple copies per round, the new copy must be melted off, typically in a Polymerase Chain Reaction (PCR). This process requires precise temperature control over the surface of the chip. Additionally, Streptavidin-Biotin bond is reversed in elevated temperatures over time [71], thus rendering PCR not suitable for on-chip reactions where the oligos are assumed to be immobilized throughout the process. Here, we build upon IPL and present a novel scheme for DNA microscopy: Chip-based Iterative Proximity Ligation and Extension (ChIPLEx), that is optimized for scale and stability on-chip, using purely isothermal reactions.

Oligonucleotide Library Preparation

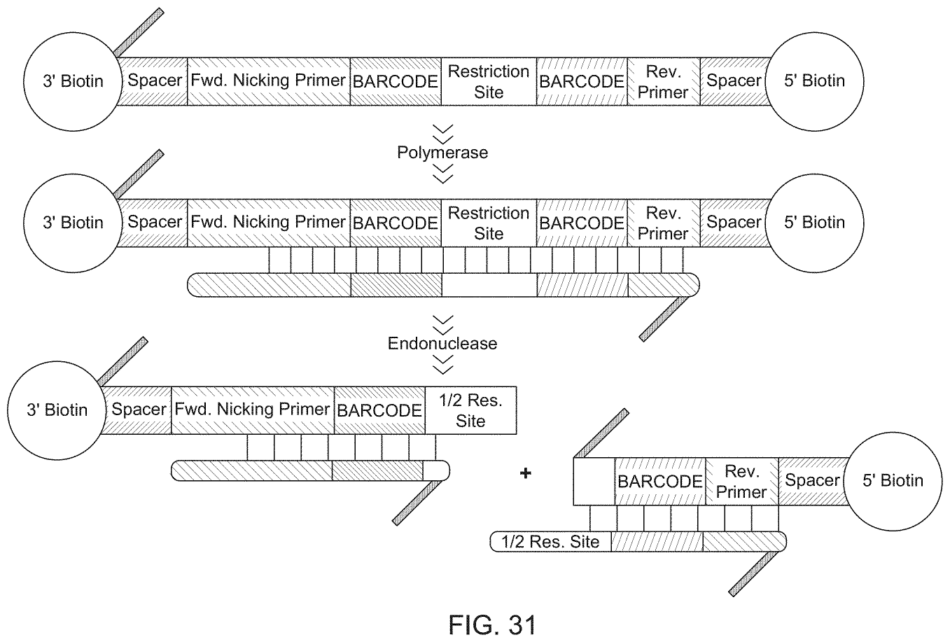

First, FIG. 31 describes the oligo design and preparation process. We start from a long ssDNA tagged with Biotin on both ends. The ssDNA is composed of two spacer domains, two unique barcode regions, two primer sites, and a single restriction site in the middle. The forward priming site contains a nicking site. To prepare the oligo library for immobilization, the ssDNA is first extended to dsDNA using a DNA Polymerase and then cleaved by a restriction enzyme (e.g., EcoRI) to produce two separate oligos—3′ Biotin and 5′ Biotin, each with a unique barcode. The restriction enzyme is chosen to produce “sticky” ends—short complementary sections that bind to each other at low affinity. Importantly, these reaction steps all happen in an aqueous solution as a preliminary step to the on-chip reactions, i.e., prior to on-chip immobilization. Last, the oligos are purified using gel electrophoresis for later suspension on the chiplet.

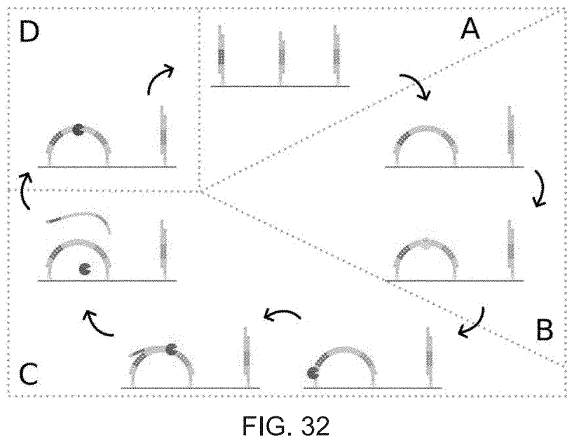

FIG. 32 illustrates an inventive Chip-based Iterative Proximity Ligation and Extension (ChIPLEx) method. In step (A), two populations of oligos, 5′ and 3′, are immobilized on-chip. Each oligo has a unique barcode, which is color-coded in FIG. 32. Step (B) illustrates sticky ends of adjacent oligos ligated utilizing a DNA Ligase (an enzyme illustrated in yellow). Step (C) illustrates On-chip Nicking Enzyme Amplification Reaction (NEAR); see reference [72]. A nicking endonuclease (not illustrated) nicks the primer site and allows DNA polymerase (an enzyme shown in red) to perform extension while displacing the previous strand. The isothermal process repeats multiple times such that multiple copies containing two barcodes are suspended in the reaction solution. The oligos are cleaved in step (D) by a restriction enzyme (shown in purple).

FIG. 33A illustrates the effects of temperature on the Biotin-Streptavidin release, varied over a temperature range from room temperature to 80° C. and a time ranging from 1 second to 5 minutes. FIG. 33B illustrates the effect of various salt concentrations at 70° C. over a short period of time (1 second). Both FIGS. 33A and 33B were adapted from [71].

FIG. 34 illustrates the effect of temperature on Streptavidin-Biotin bond with respect to various isothermal amplification buffers. The height of each column corresponds to the fluorescence intensity decrease between the chiplet suspended in the tested condition compared to a chiplet that was suspended in water at room temperature. IsoAmp (Isothermal Amplification Buffer) is optimized for Bst polymerase reactions at 50-65° C. NEBuffer 2.1 is optimized for a range of DNA polymerases and nicking endonucleases, temperatures ranging between 37-45° C. (Bsu DNA Polymerase) or room temperature (Klenow). Significant decreases occur for all buffers at elevated temperatures. Fluorescence measurements were taken using Zeiss™ LSM 5 Pascal at the Center for Bits and Atoms.

FIG. 35 illustrates the working mechanism of a Molecular Beacon (MB), which forms a stem-loop structure and thus holds the fluorophore (orange) and quencher (blue) in close proximity. Consequently, the fluorescence emission is strongly suppressed (in the absence of a target). The complementary target sequence hybridizes with the loop domain of the MB, forcing the stem helix to open, whereupon fluorescence is restored due to the spatial separation of the fluorophore from the quencher.

FIG. 36A is a Calibration curve for known concentrations of the target oligo utilizing Quantitative Nicking Enzyme Amplification Reaction (NEAR) with Molecular Beacons (MB) at a concentration of 0.2 μM. FIG. 36B is a chart of fluorescence signal-to-background ratio over time obtained with Real-time Quantitative NEAR using Molecular Beacons. The long-short-short-long broken line is a positive control for a known target oligo concentration of 1 μM. Negative controls are shown under the same conditions but lacking enzymes (long-short-long broken line) or primer (evenly dashed line). The solid line is the NEAR solution, with a starting template oligo concentration of 0.1 μM. MB concentration is 0.2 μM. Fluorescence measurements were taken using Biotek® Synergy H1 microplate reader at the Center for Bits and Atoms.

FIG. 37A illustrates a first step in Decorating a DNA Canvas. Given a pattern (top-left) and spatially located barcodes (top-right) we deduce a list of barcodes that form the pattern. As shown in FIG. 37B, complementary strands can then be conjugated with nanomaterials such as gold nano particles (shown in yellow) to form a nanoscale pattern on a DNA canvas.

FIG. 38 is a toy Example illustrating Combinatorial Decoration. In the middle, a U-shaped pattern (4×4 grid) is shown with a list of 4 base-long barcodes and their spatial locations, illustrated by their position on a grid. The pixels to be decorated are shaded. Pixels that must be “off” are in white, and wavy pixels signify “Don't Cares”, irrelevant whether the pixel is decorated or not. All distinct equivalent conformations are displayed around the perimeter of the Figure. Next to each conformation is the optimal mixed bases sequence set that would hybridize to form the pattern (while ignoring Don't Care's). The original pattern trivially requires seven sequences, most conformations require only two mixed bases sequences, and four specific conformations require just a single sequence to be synthesized. These sequences were computed by exhaustive search.

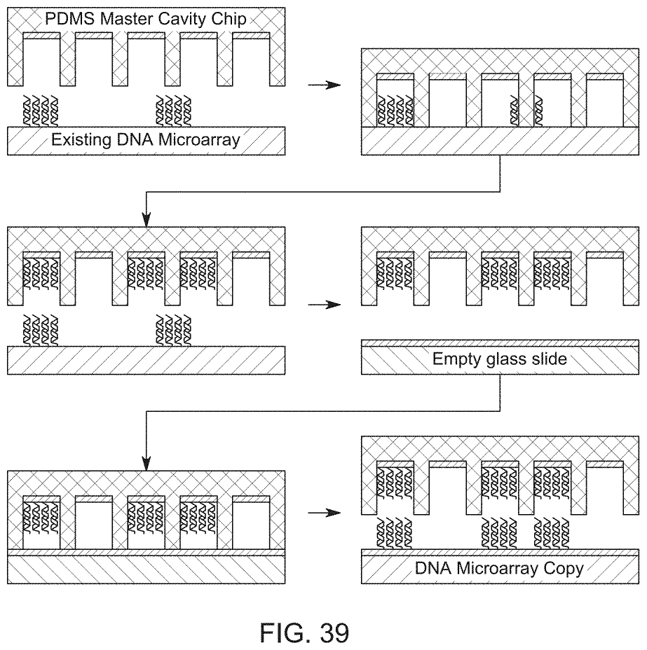

FIG. 39 illustrates the principle of DNA micro-array copying. A PDMS master cavity chip coated with primer is filled with spPCR mix and placed on top of an original DNA microarray consisting of two different DNA species (left and right). After closing, a first spPCR is performed to amplify the DNA and to attach it to the inside of the cavities of the chip. Note the “right” species attaches to two cavities. The cavity chip is washed, blocked, and refilled with fresh spPCR mix. The cavity chip is placed on top of an empty glass slide coated with primer and a second spPCR is performed. After the spPCR, the cavity chip is opened, revealing a copy of the original DNA microarray. The position, size and number of the cavities limits the spatial resolution of the copy. In this example, the left DNA spot is enclosed by one cavity resulting in one dot in the copy. The right DNA spot is enclosed by two cavities, hence creating two “right” DNA dots. Adapted from [90].

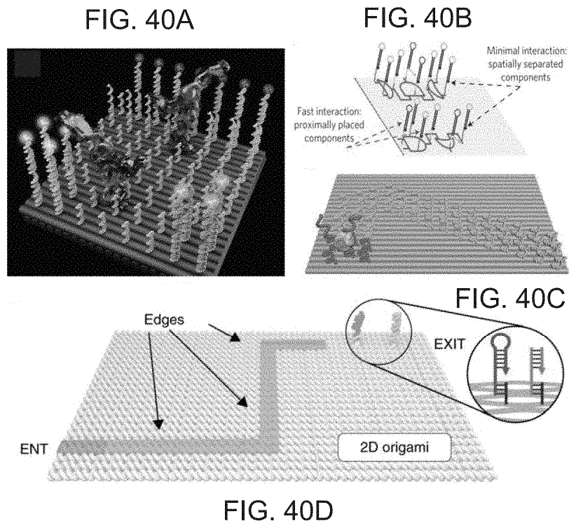

FIGS. 40A, 40B, 40C, and 40D: Conceptual illustrations of DNA robots and circuits. FIG. 40A is a conceptual illustration of DNA robots collectively transporting fluorescent cargo from an initially unsorted source to separated destinations, adapted from [96]. FIG. 40B is a conceptual illustration of a Localized DNA circuit for accelerated molecular computing, adapted from [100]. FIG. 40C is a conceptual illustration of a three-legged DNA spider that can walk along a designed track, adapted from [21]. FIG. 40D is a conceptual illustration of a DNA navigator maze setup on a DNA Origami substrate. Adapted from [97].

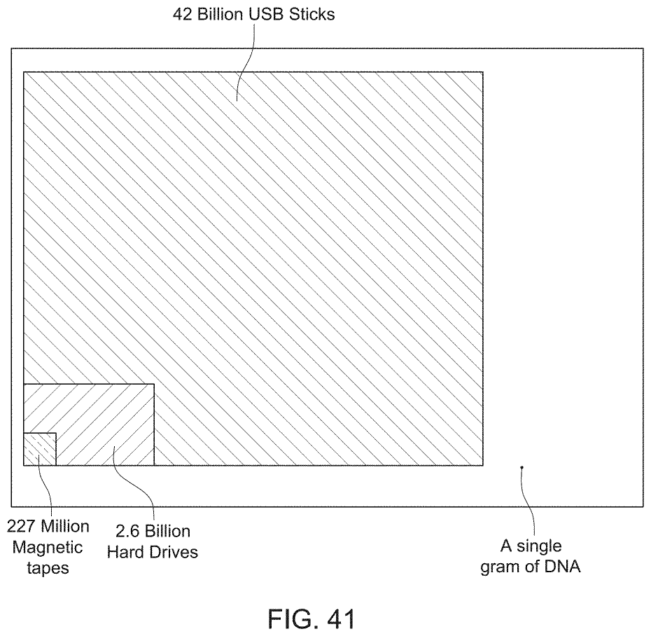

FIG. 41 is a prior art schematic adapted from [11] illustrating the amount of traditional storage media needed to store 40 Zettabytes of data versus DNA.

It should be understood, of course, that the foregoing relates to exemplary embodiments of the invention and that modifications may be made without departing from the spirit and scope of the invention as set forth in the following claims.

### BIBLIOGRAPHY

[1] Nadrian C Seeman. Nucleic acid junctions and lattices. Journal of theoretical biology, 99(2):237-247, 1982.
[2] M. C. Escher. Depth, 1955. Taken from https://www.wikiart.org/en/m-c-escher/depth. Accessed Aug. 10, 2021.
[3] Nadrian C Seeman. DNA in a material world. Nature, 421(6921):427-431, 2003.
[4] Pengfei Wang, Travis A Meyer, Victor Pan, Palash K Dutta, and Yonggang Ke. The beauty and utility of DNA origami. Chem, 2(3):359-382, 2017.
[5] Paul W K Rothemund. Folding DNA to create nanoscale shapes and patterns. Nature, 440(7082):297-302, 2006.
[6] Nadrian C Seeman and Hanadi F Sleiman. Dna nanotechnology. Nature Reviews Materials, 3(1):1-23, 2017.
[7] KA Wetterstrand. DNA sequencing costs: Data from the NHGRI genome sequencing program (GSP) available at: www.genome.gov/sequencingcosts. Accessed Aug. 1, 2021.
[8] SL Beaucage and MH Caruthers. Deoxynucleoside phosphoramidites—a new class of key intermediates for deoxypolynucleotide synthesis. Tetrahedron letters, 22(20): 1859-1862, 1981.
[9] Athel Cornish-Bowden. Nomenclature for incompletely specified bases in nucleic acid sequences: recommendations 1984. Nucleic acids research, 13(9):3021, 1985.
[10] Linda C Meiser, Julian Koch, Philipp L Antkowiak, Wendelin J Stark, Reinhard Heckel, and Robert N Grass. DNA synthesis for true random number generation. Nature communications, 11(1):1-9, 2020.
[11] Potomac Institute for Policy Studies. Future of DNA data storage. https://potomacinstitute.org/images/studies/Future_of_DNA_Data_Storage.pdf. (Online; accessed 4 Aug. 2021).
[12] Audrey Sassolas, Beatrice D Leca-Bouvier, and Loïc J Blum. DNA biosensors and microarrays. Chemical reviews, 108(1):109-139, 2008.
[13] Roger Bumgarner. Overview of DNA microarrays: types, applications, and their future. Current protocols in molecular biology, 101(1):22-1, 2013.
[14] Henrik Bengtsson and Terry Speed. Copy-number estimation using robust multichip analysis, 2007. (Supplementary materials for the aroma.affymetrix lab session).
[15] Stephen P Fodor, J Leighton Read, Michael C Pirrung, Lubert Stryer, A Tsai Lu, and Dennis Solas. Light-directed, spatially addressable parallel chemical synthesis. science, 251(4995):767-773, 1991.
[16] Frank F Bier and Frank Kleinjung. Feature-size limitations of microarray technology—a critical review. Fresenius' journal of analytical chemistry, 371(2):151-156, 2001.
[17] Abhijit Biswas, Ilker S Bayer, Alexandru S Biris, Tao Wang, Enkeleda Dervishi, and Franz Faupel. Advances in top-down and bottom-up surface nanofabrication: Techniques, applications & future prospects. Advances in colloid and interface science, 170(1-2):2-27, 2012.
[18] Anirban Samanta and Igor L Medintz. Nanoparticles and DNA—a powerful and growing functional combination in bionanotechnology. Nanoscale, 8(17):9037-9095, 2016.
[19] Hari K K Subramanian, Banani Chakraborty, Ruojie Sha, and Nadrian C Seeman. The label-free unambiguous detection and symbolic display of single nucleotide polymorphisms on DNA origami. Nano letters, 11(2):910-913, 2011.
[20] Rahul Chhabra, Jaswinder Sharma, Yonggang Ke, Yan Liu, Sherri Rinker, Stuart Lindsay, and Hao Yan. Spatially addressable multiprotein nanoarrays templated by aptamer-tagged DNA nanoarchitectures. Journal of the American Chemical Society, 129(34): 10304-10305, 2007.
[21] Fan Hong, Fei Zhang, Yan Liu, and Hao Yan. DNA origami: scaffolds for creating higher order structures. Chemical reviews, 117(20):12584-12640, 2017.
[22] Ryan J Kershner, Luisa D Bozano, Christine M Micheel, Albert M Hung, Ann R Fornof, Jennifer N Cha, Charles T Rettner, Marco Bersani, Jane Frommer, Paul W K Rothemund, et al. Placement and orientation of individual dna shapes on lithographically patterned surfaces. Nature Nanotechnology, 4(9):557-561, 2009.
[23] Ashwin Gopinath and Paul W K Rothemund. Optimized assembly and covalent coupling of single-molecule dna origami nanoarrays. Acs Nano, 8(12):12030-12040, 2014.
[24] Ashwin Gopinath, Evan Miyazono, Andrei Faraon, and Paul W K Rothemund. Engineering and mapping nanocavity emission via precision placement of dna origami. Nature, 535(7612):401-405, 2016.
[25] Rishabh M Shetty, Sarah R Brady, Paul W K Rothemund, Rizal F Hariadi, and Ashwin Gopinath. Bench-top fabrication of single-molecule nanoarrays by dna origami placement. ACS nano, page 250951, 2021.
[26] Grigory Tikhomirov, Philip Petersen, and Lulu Qian. Fractal assembly of micrometre-scale DNA origami arrays with arbitrary patterns. Nature, 552(7683):67-71, 2017.
[27] Alexander A Boulgakov, Andrew D Ellington, and Edward M Marcotte. Bringing microscopy-by-sequencing into view. Trends in biotechnology, 38(2):154-162, 2020.
[28] [28] Joshua A Weinstein, Aviv Regev, and Feng Zhang. DNA microscopy: optics-free spatio-genetic imaging by a stand-alone chemical reaction. Cell, 178(1):229-241, 2019.
[29] Joshua I Glaser, Bradley M Zamft, George M Church, and Konrad P Kording. Puzzle imaging: Using large-scale dimensionality reduction algorithms for localization. PloS one, 10(7):e0131593, 2015.
[30] Ian T Hoffecker, Yunshi Yang, Giulio Bernardinelli, Pekka Orponen, and Björn Högberg. A computational framework for DNA sequencing-based microscopy. bioRxiv, page 476200, 2018.
[31] Patrick S Stayton, Stefanie Freitag, Lisa A Klumb, Ashutosh Chilkoti, Vano Chu, Julie E Penzotti, Richard To, David Hyre, Isolde Le Trong, Terry P Lybrand, et al. Streptavidin-biotin binding energetics. Biomolecular engineering, 16(1-4):39-44, 1999.
[32] Akinori Kuzuya, Kentaro Numajiri, Mayumi Kimura, and Makoto Komiyama. Single-molecule accommodation of streptavidin in nanometer-scale wells formed in DNA nanostructures. In Nucleic Acids Symposium Series, volume 52, pages 681-682. Oxford University Press, 2008.
[33] Bruce A Hendrickson. The molecule problem: Determining conformation from pairwise distances. Technical report, Cornell University, 1990.
[34] Amit Singer. A remark on global positioning from local distances. Proceedings of the National Academy of Sciences, 105(28):9507-9511, 2008.
[35] Paul Erdös and Alfred Rényi. On the strength of connectedness of a random graph. Acta Mathematica Hungarica, 12(1):261-267, 1961.
[36] [36] Stephen G Kobourov. Spring embedders and force directed graph drawing algorithms. arXiv preprint arXiv:1201.3011, 2012.
[37] Thomas M J Fruchterman and Edward M Reingold. Graph drawing by force-directed placement. Software: Practice and experience, 21(11):1129-1164, 1991.
[38] Tomihisa Kamada, Satoru Kawai, et al. An algorithm for drawing general undirected graphs. Information processing letters, 31(1):7-15, 1989.
[39] Chris Walshaw et al. A multilevel algorithm for force-directed graph-drawing. Journal of Graph Algorithms and Applications, 7(3):253-285, 2006.
[40] Yifan Hu. Efficient, high-quality force-directed graph drawing. Mathematica Journal, 10(1):37-71, 2005.
[41] [41] Josh Barnes and Piet Hut. A hierarchical O (N log N) force-calculation algorithm. nature, 324(6096):446-449, 1986.
[42] David Harel and Yehuda Koren. Graph drawing by high-dimensional embedding. In International symposium on graph drawing, pages 207-219. Springer, 2002.
[43] Wolfgang Kabsch. A discussion of the solution for the best rotation to relate two sets of vectors. Acta Crystallographica Section A: Crystal Physics, Diffraction, Theoretical and General Crystallography, 34(5):827-828, 1978.
[44] David Sims, Ian Sudbery, Nicholas E Ilott, Andreas Heger, and Chris P Ponting. Sequencing depth and coverage: key considerations in genomic analyses. Nature Reviews Genetics, 15(2):121-132, 2014.
[45] Vilfredo Pareto. Cours d'oconomie politique, volume 1. Librairie Droz, 1964.
[46] Tiago P. Peixoto. The graph-tool Python library. figshare, 2014.
[47] Charles R. Harris, K. Jarrod Millman, Stefan J. van der Walt, Ralf Gommers, Pauli Virtanen, David Cournapeau, Eric Wieser, Julian Taylor, Sebastian Berg, Nathaniel J. Smith, Robert Kern, Matti Picus, Stephan Hoyer, Marten H. van Kerkwijk, Matthew Brett, Allan Haldane, Jaime Fernandez del Rio, Mark Wiebe, Pearu Peterson, Pierre Gérard-Marchant, Kevin Sheppard, Tyler Reddy, Warren Weckesser, Hameer Abbasi, Christoph Gohlke, and Travis E. Oliphant. Array programming with NumPy. Nature, 585(7825):357-362, September 2020.
[48] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, and E. Duchesnay. Scikit-learn: Machine learning in Python. Journal of Machine Learning Research, 12:2825-2830, 2011.
[49] [49] J. D. Hunter. Matplotlib: A 2D graphics environment. Computing in Science & Engineering, 9(3):90-95, 2007.
[50] Michael L. Waskom. seaborn: statistical data visualization. Journal of Open Source Software, 6(60):3021, 2021.
[51] Osamu Yogi, Tomonori Kawakami, Masayo Yamauchi, Jing Yong Ye, and Mitsuru Ishikawa. On-demand droplet spotter for preparing pico-to femtoliter droplets on surfaces. Analytical chemistry, 73(8):1896-1902, 2001.
[52] Yanzhen Zhang, Benliang Zhu, Yonghong Liu, and Gunther Wittstock. Hydrodynamic dispensing and electrical manipulation of attolitre droplets. Nature communications, 7(1):1-7, 2016.
[53] Michael C Pirrung. How to make a DNA chip. Angewandte Chemie International Edition, 41(8):1276-1289, 2002.
[54] Wan Zhou, Mengli Feng, Alejandra Valadez, and XiuJun Li. One-step surface modification to graft DNA codes on paper: The method, mechanism, and its application. Analytical Chemistry, 92(10):7045-7053, 2020. PMID: 32207965.
[55] Satish Balasaheb Nimse, Keumsoo Song, Mukesh Digambar Sonawane, Danishmalik Rafiq Sayyed, and Taisun Kim. Immobilization techniques for microarray: challenges and applications. Sensors, 14(12):22208-22229, 2014.
[56] Christopher M Dundas, Daniel Demonte, and Sheldon Park. Streptavidin-biotin technology: improvements and innovations in chemical and biological applications. Applied microbiology and biotechnology, 97(21):9343-9353, 2013.
[57] Ángel Rios, Mohammed Zougagh, and Monica Avila. Miniaturization through lab-on-a-chip: Utopia or reality for routine laboratories? a review. Analytica chimica acta, 740:1-11, 2012.
[58] Céline Adessi, Gilles Matton, Guidon Ayala, Gerardo Turcatti, Jean-Jacques Mermod, Pascal Mayer, and Eric Kawashima. Solid phase DNA amplification: characterisation of primer attachment and amplification mechanisms. Nucleic acids research, 28(20):e87-e87, 2000.
[59] Omar Bagasra. Protocols for the in situ PCR-amplification and detection of mRNA and DNA sequences. Nature protocols, 2(11):2782-2795, 2007.
[60] Zeno Guttenberg, Helena Müller, Heiko Habermüller, Andreas Geisbauer, Jürgen Pipper, Jana Felbel, Mark Kielpinski, Jürgen Scriba, and Achim Wixforth. Planar chip device for PCR and hybridization with surface acoustic wave pump. Lab on a Chip, 5(3):308-317, 2005.
[61] Matthew D Estes, Jianing Yang, Brett Duane, Stan Smith, Carla Brooks, Alan Nordquist, and Frederic Zenhausern. Optimization of multiplexed PCR on an integrated microfluidic forensic platform for rapid DNA analysis. Analyst, 137(23):5510-5519, 2012.
[62] Rabih Zaouk, Benjamin Y Park, and Marc J Madou. Introduction to microfabrication techniques. In Microfluidic Techniques, pages 5-15. Springer, 2006.
[63] José M. Quero, Francisco Perdigones, and Carmen Aracil. 11-microfabrication technologies used for creating smart devices for industrial applications. In Stoyan Nihtianov and Antonio Luque, editors, Smart Sensors and MEMs (Second Edition), Woodhead Publishing Series in Electronic and Optical Materials, pages 291-311. Woodhead Publishing, second edition edition, 2018.
[64] C J Mogab, A C Adams, and D L Flamm. Plasma etching of Si and SiO₂—the effect of oxygen additions to CF4 plasmas. Journal of applied physics, 49(7):3796-3803, 1978.
[65] Amid Shakeri, Noor Abu Jarad, Ashlyn Leung, Leyla Soleymani, and Tohid F Didar. Biofunctionalization of glass- and paper-based microfluidic devices: A review. Advanced Materials Interfaces, 6(19):1900940, 2019.
[66] Elena Ambrosetti, Giulio Bernardinelli, Ian Hoffecker, Leonard Hartmanis, Georges Kiriako, Ario De Marco, Rickard Sandberg, Björn Högberg, and Ana I Teixeira. A DNA-nanoassembly-based approach to map membrane protein nanoenvironments. Nature Nanotechnology, 16(1):85-95, 2021.
[67] Thomas E Schaus, Sungwook Woo, Feng Xuan, Xi Chen, and Peng Yin. A DNA nanoscope via auto-cycling proximity recording. Nature communications, 8(1):1-9, 2017.
[68] Xin Song and John Reif. Optics-free imaging with DNA microscopy: An overview, 2021.
[69] Alexander A Boulgakov, Erhu Xiong, Sanchita Bhadra, Andrew D Ellington, and Edward M Marcotte. From space to sequence and back again: Iterative DNA proximity ligation and its applications to DNA-based imaging. bioRxiv, page 470211, 2018.
[70] Nikhil Gopalkrishnan, Sukanya Punthambaker, Thomas E Schaus, George M Church, and Peng Yin. A DNA nanoscope that identifies and precisely localizes over a hundred unique molecular features with nanometer accuracy. bioRxiv, 2020.
[71] Anders Holmberg, Anna Blomstergren, Olof Nord, Morten Lukacs, Joakim Lundeberg, and Mathias Uhlén. The biotin-streptavidin interaction can be reversibly broken using water at elevated temperatures. Electrophoresis, 26(3):501-510, 2005.
[72] Cheng Qian, Rui Wang, Hui Wu, Feng Ji, and Jian Wu. Nicking enzyme-assisted amplification (NEAA) technology and its applications: a review. Analytica chimica acta, 1050:1-15, 2019.
[73] [73] Brian K Maples, Rebecca C Holmberg, Andrew P Miller, Jarrod W Provins, Richard B Roth, and Jeffrey G Mandell. Nicking and extension amplification reaction for the exponential amplification of nucleic acids, Jun. 27, 2017. U.S. Pat. No. 9,689,031.
[74] M Dean Savage. Avidin-biotin chemistry. Pierce Chemical Co., 1992.
[75] Xinchun Tong and Lloyd Smith. Solid-phase method for the purification of DNA sequencing reactions. Analytical chemistry, 64(22):2672-2677, 1992.
[76] Tsugunori Notomi, Hiroto Okayama, Harumi Masubuchi, Toshihiro Yonekawa, Keiko Watanabe, Nobuyuki Amino, and Tetsu Hase. Loop-mediated isothermal amplification of DNA. Nucleic Acids Research, 28(12):e63-e63, June 2000.
[77] Liu Wang, Cheng Qian, Hui Wu, Wenjuan Qian, Rui Wang, and Jian Wu. Technical aspects of nicking enzyme assisted amplification. Analyst, 143(6):1444-1453, 2018.
[78] Markus von Nickisch-Rosenegk, Xenia Marschan, Dennie Andresen, Alexandra Abraham, Christian Heise, and Frank F Bier. On-chip PCR amplification of very long templates using immobilized primers on glassy surfaces. Biosensors and Bioelectronics, 20(8):1491-1498, 2005.
[79] Jochen Hoffmann, Sebastian Hin, Felix von Stetten, Roland Zengerle, and Günter Roth. Universal protocol for grafting PCR primers onto various lab-on-a-chip substrates for solid-phase PCR. Rsc Advances, 2(9):3885-3889, 2012.
[80] MS Shchepinov, S C Case-Green, and EM Southern. Steric factors influencing hybridisation of nucleic acids to oligonucleotide arrays. Nucleic acids research, 25(6):1155-1161, 1997.
[81] A Carmon, T J Vision, S E Mitchell, T W Thannhauser, U Müller, and S Kresovich. Solid-phase PCR in microwells: effects of linker length and composition on tethering, hybridization, and extension. BioTechniques, 32(2):410-420, 2002.
[82] Mark E Fornace, Nicholas J Porubsky, and Niles A Pierce. A unified dynamic programming framework for the analysis of interacting nucleic acid strands: Enhanced models, scalability, and speed. ACS Synthetic Biology, 9(10):2665-2678, 2020.
[83] Kem in Wang, Zhiwen Tang, Chaoyong James Yang, Youngmi Kim, Xiaohong Fang, Wei Li, Yanrong Wu, Colin D Medley, Zehui Cao, Jun Li, et al. Molecular engineering of DNA: molecular beacons. Angewandte Chemie International Edition, 48(5):856-870, 2009.
[84] [84] Weihong Tan, Kemim Wang, and Timothy J Drake. Molecular beacons. Current opinion in chemical biology, 8(5):547-553, 2004.
[85] Jacqueline A M Vet and Salvatore A E Marras. Design and optimization of molecular beacon real-time polymerase chain reaction assays. In Oligonucleotide Synthesis, pages 273-290. Springer, 2005.
[86] [86] Wei Liu, Simo Huang, Ningwei Liu, Derong Dong, Zhan Yang, Yue Tang, Wen Ma, Xiaoming He, Da Ao, Yaqing Xu, et al. Establishment of an accurate and fast detection method using molecular beacons in loop-mediated isothermal amplification assay. Scientific reports, 7(1):1-9, 2017.
[87] Anton Kuzyk, Ralf Jungmann, Guillermo P Acuna, and Na Liu. DNA origami route for nanophotonics. ACS photonics, 5(4):1151-1163, 2018.
[88] Yuri L Lyubchenko, Luda S Shlyakhtenko, and Toshio Ando. Imaging of nucleic acids with atomic force microscopy. Methods, 54(2):274-283, 2011.
[89] Maurice Karnaugh. The map method for synthesis of combinational logic circuits. Transactions of the American Institute of Electrical Engineers, Part I: Communication and Electronics, 72(5):593-599, 1953.
[90] Stefan D Krämer, Johannes Wöhrle, Philipp A Meyer, Gerald A Urban, and Günter Roth. How to copy and paste DNA microarrays. Scientific reports, 9(1):1-10, 2019.
[91] David J Lockhart, Helin Dong, Michael C Byrne, Maximillian T Follettie, Michael V Gallo, Mark S Chee, Michael Mittmann, Chunwei Wang, Michiko Kobayashi, Heidi Norton, et al. Expression monitoring by hybridization to high-density oligonucleotide arrays. Nature biotechnology, 14(13):1675-1680, 1996.
[92] Fei Chen, Paul W Tillberg, and Edward S Boyden. Expansion microscopy. Science, 347(6221):543-548, 2015.
[93] Kyle Lund, Anthony J Manzo, Nadine Dabby, Nicole Michelotti, Alexander Johnson-Buck, Jeanette Nangreave, Steven Taylor, Renjun Pei, Milan N Stojanovic, Nils G Walter, et al. Molecular robots guided by prescriptive landscapes. Nature, 465(7295):206-210, 2010.
[94] Eric Bonabeau, Marco Dorigo, and Guy Theraulaz. Inspiration for optimization from social insect behaviour. Nature, 406(6791):39-42, 2000.
[95] Hongzhou Gu, Jie Chao, Shou-Jun Xiao, and Nadrian C Seeman. A proximity-based programmable DNA nanoscale assembly line. Nature, 465(7295):202-205, 2010.
[96] Anupama J Thubagere, Wei Li, Robert F Johnson, Zibo Chen, Shayan Doroudi, Yae Lim Lee, Gregory Izatt, Sarah Wittman, Niranjan Srinivas, Damien Woods, et al. A cargo-sorting DNA robot. Science, 357(6356), 2017.
[97] Jie Chao, Jianbang Wang, Fei Wang, Xiangyuan Ouyang, Enzo Kopperger, Huajie Liu, Qian Li, Jiye Shi, Lihua Wang, Jun Hu, et al. Solving mazes with single-molecule DNA navigators. Nature materials, 18(3):273-279, 2019.
[98] Georg Seelig, David Soloveichik, David Yu Zhang, and Erik Winfree. Enzyme-free nucleic acid logic circuits. science, 314(5805):1585-1588, 2006.
[99] Lulu Qian, Erik Winfree, and Jehoshua Bruck. Neural network computation with dna strand displacement cascades. Nature, 475(7356):368-372, 2011.
[100] Gourab Chatterjee, Neil Dalchau, Richard A Muscat, Andrew Phillips, and Georg Seelig. A spatially localized architecture for fast and modular dna computing. Nature nanotechnology, 12(9):920-927, 2017.
[101] Yiming Dong, Fajia Sun, Zhi Ping, Qi Ouyang, and Long Qian. DNA storage: research landscape and future prospects. National Science Review, 7(6):1092-1107, 2020.
[102] Andy Extance. How DNA could store all the world's data. Nature News, 537(7618):22, 2016.
[103] Joe Davis. Microvenus. Art Journal, 55(1):70-74, 1996.
[104] Lee Organick, Siena Dumas Ang, Yuan-Jyue Chen, Randolph Lopez, Sergey Yekhanin, Konstantin Makarychev, Miklos Z Racz, Govinda Kamath, Parikshit Gopalan, Bichlien Nguyen, et al. Random access in large-scale DNA data storage. Nature biotechnology, 36(3):242-248, 2018.
[105] Luis Ceze, Jeff Nivala, and Karin Strauss. Molecular digital data storage using DNA. Nature Reviews Genetics, 20(8):456-466, 2019.
[106] Martin G T A Rutten, Frits W Vaandrager, Johannes A A W Elemans, and Roeland J M Nolte. Encoding information into polymers. Nature Reviews Chemistry, 2(11):365-381, 2018.
[107] Randall A Hughes and Andrew D Ellington. Synthetic DNA synthesis and assembly: putting the synthetic in synthetic biology. Cold Spring Harbor perspectives in biology, 9(1):a023812, 2017.
[108] Henry H Lee, Reza Kalhor, Naveen Goela, Jean Bolot, and George M Church. Terminator-free template-independent enzymatic DNA synthesis for digital information storage. Nature communications, 10(1):1-12, 2019.
[109] Robert E Fontana Jr and Gary M Decad. Moore's law realities for recording systems and memory storage components: HDD, tape, NAND, and optical. AIP Advances, 8(5):056506, 2018.

What is claimed is:

1. A DNA nanoarray, comprising:
a milliscale chip substrate;
a binder bound to the milliscale chip substrate as a microscale spot having a uniform surface; and
immobilized oligonucleotide sequences, each having a linker linked to the binder such that the immobilized oligonucleotide sequences form a monolayer, each of the immobilized oligonucleotide sequences having a length of at least N, wherein N is a minimum length operative to guarantee within a statistical certainty that the immobilized oligonucleotide sequences are each unique.

2. The DNA nanoarray of claim 1, further comprising a data set having a 2D coordinate indicating a location of at least a sample of the immobilized oligonucleotide sequences on the uniform surface.

3. The DNA nanoarray of claim 1 wherein the linker comprises biotin and the binder comprises streptavidin.

4. The DNA nanoarray of claim 1, wherein the immobilized oligonucleotide sequences are present in a monolayer grid topology.

5. The DNA nanoarray of claim 1, wherein the microscale spot is surrounded by alignment markers.

6. The DNA nanoarray of claim 1, wherein the microscale spot has a diameter less than about 10 μm.

7. A platform for DNA information storage, comprising the DNA nanoarray of claim 1.

8. A platform for digital information storage, comprising the DNA nanoarray of claim 1.

9. The DNA nanoarray of claim 1, wherein the immobilized oligonucleotide sequences are each operative as a uniquely addressable DNA nanopixel.

10. A method of producing a DNA nanoarray, comprising:
providing a streptavidin-coated substrate;
patterning the streptavidin-coated substrate by photolithography to produce a patterned surface having an array of microscale spots with active streptavidin binding sites; and
immobilizing biotin-tagged oligonucleotides on the patterned surface by applying a solution containing the biotin-tagged oligonucleotides to the array of microscale spots; applying a buffer over the patterned surface; and washing the patterned surface in buffered saline solution;
wherein the biotin-tagged oligonucleotides each have a string of bases with a length operative to guarantee within a statistical certainty that the string of bases of the immobilized biotin-tagged oligonucleotide are each unique.

11. The method of claim 10, further comprising preparing the biotin-tagged oligonucleotides prior to immobilizing, including:
providing at least one single-stranded DNA (ssDNA) in an aqueous solution, the at least one ssDNA having two spacer domains tagged with biotin on each end, with a forward priming site having a nicking site, a first unique barcode region having a first unique string of bases, a restriction site in a center of the at least one ssDNA, a second unique barcode region having a second unique string of bases, and a reverse primer site therebetween;
extending the at least one ssDNA to a double-stranded DNA (dsDNA) by a polymerase reaction;
cleaving the dsDNA with a restriction enzyme to produce a 3′ biotin oligonucleotide having the first unique barcode region and a 5′ biotin oligonucleotide having the second unique barcode region; and
separating the 3′ biotin oligonucleotide and the 5′ biotin oligonucleotide by gel electrophoresis.

12. The method of claim 10, further comprising:
separating the streptavidin-coated substrate into chiplets dimensioned to fit within a PCR tube;
preparing more than one of the PCR tube, each containing a liquid volume comprising buffers and enzymes selected for a specific co-localized enzymatic reaction selected from the group consisting of ligation, amplification, and restriction;
introducing the chiplet sequentially into the more than one of the PCR tube, thereby generating a pool of ligation events representing local proximity information about the immobilized biotin-tagged oligonucleotides; and
sequencing the pool of ligation events.

13. The method of claim 10, further comprising prior to the step of immobilizing the biotin-tagged oligonucleotides:
applying a positive resist to the patterned surface;
exposing and developing the patterned surface;
etching optically visible alignment markers around the microscale spots with a reactive ion; and
stripping the positive resist.

14. The method of claim 10, further comprising mapping each string of bases to a spatial two-dimensional coordinate representing the DNA nanoarray.

15. The method of claim 10, further comprising:
validating quality of the DNA nanoarray by:
synthesizing a pool of a validation oligonucleotide comprising a spacer, a priming site, and a sequence of multiple bases simulating a barcode region, wherein the bases are selected from the group consisting of A, C, and T, such that a 3′ end of the validation oligonucleotide comprises guanine or cytosine;
immobilizing the pool of the validation oligonucleotide to the patterned surface by self-assembly in a uniform monolayer;
reacting the immobilized pool of the validation oligonucleotides with DNA polymerase to perform enzymatic extension, wherein amounts of dATP, dGTP, dTTP, and dCTP-Cy3 are supplied without dCTP;
washing the patterned surface;
applying a pool of complementary oligosaccharides, containing a sequence complementary to the sequence of the validation oligonucleotide and a fluorophore, on the patterned surface; and
observing the patterned surface with a fluorescence microscope.

16. The method of claim 15, wherein the fluorophore is selected from the group consisting of fluorescein and Cy3.

17. A method of storing information on and retrieving information from the DNA nanoarray of claim 9, comprising:
providing the DNA nanoarray;
writing bits to and/or storing spatial patterns to a subset of the nanopixels on the DNA nanoarray; and
reading and/or visualizing the bits and/or the spatial patterns.

18. The method of claim 17, further comprising defining a subset of the nanopixels that are irrelevant to the spatial pattern or do not appear on the DNA nanoarray.

19. The method of claim 17, wherein the step of reading and/or visualizing comprises optically reading microscale features with a fluorescence microscope.

20. The method of claim 17, wherein the step of reading and/or visualizing comprises reading nanoscale features and visualizing holographic elements by nanoscale topological imaging with atomic force microscopy.

21. The method of claim 17, wherein the step of writing bits and/or storing spatial patterns is performed by disabling a plurality of the nanopixels with electron beam lithography.

22. The method of claim 21, further comprising producing a copy that contains the disabled plurality of the nanopixels.

23. The method of claim 17, wherein the step of writing bits and/or storing spatial patterns is performed by photolithography, using a predetermined optical mask with a mask aligner.

24. The method of claim 17, wherein the step of writing bits and/or storing spatial patterns is performed by complementary strand hybridization.

25. The method of claim 24, wherein the complementary strands are conjugated with nanomaterials, forming a nanoscale pattern on the DNA nanoarray.
